SEQUENCE LISTING (21 sequences)

SEQ ID NO: 1 - protein, 4 aa, Artificial Sequence
  Synthetic peptide
  MISC_FEATURE (1)..(1): Xaa = N,N-Dimethyl glycine
XAPA

SEQ ID NO: 2 - protein, 4 aa, Artificial Sequence
  Synthetic peptide
  MISC_FEATURE (1)..(1): Xaa = N,N-Dimethyl glycine
XAPV

SEQ ID NO: 3 - protein, 4 aa, Artificial Sequence
  Synthetic peptide
  MISC_FEATURE (1)..(1): Xaa = N,N-Dimethyl glycine
XVPA

SEQ ID NO: 4 - protein, 4 aa, Artificial Sequence
  Synthetic peptide
  MISC_FEATURE (1)..(1): Xaa = N,N-Dimethyl glycine
XVPV

SEQ ID NO: 5 - protein, 4 aa, Artificial Sequence
  Synthetic peptide
  MISC_FEATURE (1)..(1): Xaa = Cysteic acid
XADA

SEQ ID NO: 6 - protein, 4 aa, Artificial Sequence
  Synthetic peptide
  MISC_FEATURE (1)..(1): Xaa = Cysteic acid
XADV

SEQ ID NO: 7 - protein, 4 aa, Artificial Sequence
  Synthetic peptide
  MISC_FEATURE (1)..(1): Xaa = Cysteic acid
XVDA

SEQ ID NO: 8 - protein, 4 aa, Artificial Sequence
  Synthetic peptide
  MISC_FEATURE (1)..(1): Xaa = Cysteic acid
XVDV

SEQ ID NO: 9-10 - skipped (2 entries)

SEQ ID NO: 11 - DNA, 29 bp, Artificial Sequence
  Synthetic oligonucleotide
gtatgcacctgggctctttgcaggtctct

SEQ ID NO: 12-15 - skipped (4 entries)

SEQ ID NO: 16 - DNA, 29 bp, Artificial Sequence
  Synthetic oligonucleotide
gtatgcacctgggctctttgcaggtctct

SEQ ID NO: 17 - DNA, 29 bp, Artificial Sequence
  Synthetic oligonucleotide
tctctggacgtttctcgggtccacgtatg

SEQ ID NO: 18 - DNA, 60 bp, Artificial Sequence
  Synthetic oligonucleotide
aggggtttgctccggagagacctgcaaagagcccaggtgcataccttggcaatctgcata

SEQ ID NO: 19-20 - skipped (2 entries)

SEQ ID NO: 21 - DNA, 29 bp, Artificial Sequence
  Synthetic oligonucleotide
gtatgcacctgggctctttgcaggtctct

(19) United States
(12) Patent Application Publication
(10) Pub. No.: US 2011/0172115 A1
(43) Pub. Date: Jul. 14, 2011

(54) CHARACTERISING PLANAR SAMPLES BY MASS SPECTROMETRY

(76) Inventor: Andrew H. Thompson, Cambridge (GB)

(21) Appl. No.: 13/001,232

(22) PCT Filed: Jun. 24, 2009
(86) PCT No.: PCT/GB2009/001583

§ 371 Date: Mar. 8, 2011

(30) Foreign Application Priority Data
Jun. 24, 2008 (GB) 0811574.3
Dec. 1, 2008 (GB) 0821945.3

(51) Int. Cl. C40B 30/04; G01N 23/00; C07K 2/00; C40B 40/10; C07H 21/00; C07K 19/00

(52) U.S. Cl. 506/9; 436/501; 435/7.1; 530/300; 530/350; 530/322; 506/18; 536/23.1; 530/391.3

(57) ABSTRACT

The present invention is directed to probes and methods for the imaging of specimens such as tissue samples or other biological specimens or arrays using mass spectrometry (MS). In one aspect, there is provided a method for analysing a specimen comprising the steps of contacting the specimen with probe molecules each of which includes one or more mass tags coupled to each probe molecule via a first cleavable linker, contacting the specimen with a Matrix-assisted Laser Desorption lonisation (MALDI) matrix or similar material, irradiating a portion of the specimen with a laser beam to release ions from the specimen, selecting released ions with mass-to-charge ratios corresponding to the mass tags or derivatives of the mass tags, and recording the amount and type of ions selected together with the location of the portion of the specimen. The irradiation, selection and recording steps are then repeated on a different portion of the specimen.

The present invention relates to probes and methods for the imaging of specimens using mass spectrometry (MS). The probes and methods are particularly suited to imaging of tissue samples or other biological specimens or arrays.

Imaging of gene and protein expression directly in tissues is a highly informative way to determine gene function and activity. Various techniques have been developed for this purpose but most current techniques are limited by the number of tags (also known as “markers”) available, restricting the number of species that can be analysed simultaneously.

Autoradiography of “in situ” hybridised probes is a well-established technique for imaging of expression patterns in tissue sections (see Wilson et al., 1997, Brain Res Brain Res Protoc. 1(2):175-85; Steel et al., 1998, Eur J Histochem. 42(2):143-50) but typically only one radiolabel can be used at a time and there are safety issues involved with the use of radiolabelled probes. Chemiluminescence and enzyme-linked probes offer safety enhancements compared to radiolabelled techniques but again the number of probes that can be resolved simultaneously is severely limited (Viale et al., 1992, Liver. 12(4 Pt 2): 243-51).

Imaging mass spectrometry (IMS) is an emerging technique in mass spectrometry with many applications, including the analysis of tissue samples. IMS offers the ability to visualise the spatial distribution of drugs in their correct physiological setting without requiring radio-labelled drugs while also being able to distinguish drug metabolites simultaneously. Drug cocktails can also be analysed by IMS, as each compound can be individually resolved.

IMS also has the potential to simultaneously analyse nucleic acids, peptides and proteins along with endogenous metabolites, opening the possibility of relating drug action to changes in the molecular profiles of tissue components such as mRNA and allowing meaningful mode of action studies to be performed and off-target activities to be determined. Understanding of developmental biology will also be enhanced by the ability to image nucleic acid and polypeptide activities in a spatially resolved manner.

However, identification of large molecular species, such as expressed mRNAs or polypeptides, by mass alone is not trivial as sensitivity of mass spectrometry to large molecules, for example large biological polymers, is currently limited.

A recent publication by the present inventor using electrospray based analysis of mass tags suggests that MS/MS based tag designs can be used to overcome background noise and give sensitive and quantitative measurements of biomolecules linked to the mass tags (Thompson et al., 2007, Nucleic Acids Res. 35(4):e28).

However, the prior art does not disclose techniques that effectively employ the capability of MS such as Matrix-assisted Laser Desorption Ionisation (MALDI) Tandem Mass Spectrometry (together termed “MALDI MS/MS”) to analyse and effectively quantify nucleic acids or polypeptides of interest as a function of their position in a test sample.

In addition, the prior art does not provide probe molecules suitable for imaging tissue samples using MS such as MALDI MS/MS.

Moreover, the prior art does not provide methods to speed up analysis of tissue samples, since it is currently very time-consuming to acquire a large number of mass spectra that are needed to produce an image from a tissue section using a mass spectrometer.

According to the present invention there is provided in one aspect a method for analysing a specimen, comprising the steps of:
a) contacting the specimen with probe molecules, each of the probe molecules including one or more mass tags coupled to each probe molecule via a first cleavable linker, and allowing probe molecules to become bound to the specimen;
b) removing unbound probe molecules from the specimen;
c) contacting the specimen with a MALDI matrix material (or MS-compatible matrix material), or laying the specimen on a surface coated with a MALDI matrix material (or MS-compatible matrix material);
d) irradiating a portion of the specimen with a laser beam to release ions from the specimen;
e) selecting, from the ions released in step (d), ions with mass-to-charge ratios corresponding to the mass tags or derivatives of the mass tags;
f) recording the amount and type of ions selected in step (e), together with the location of the portion of the specimen, as a result;
g) repeating steps (d) to (f) on a different portion of the specimen.

Further aspects and embodiments of the invention are set out in the appended claims and/or described below.

MALDI MS/MS is versatile in its many applications to the analysis of biological samples, especially to peptides and proteins. Typically, samples are mixed with an organic compound, which acts as a matrix to facilitate desorption and ionisation of compounds in the sample. The matrix provides the required sensitivity and specificity for use of laser desorption techniques in the analysis of biological material. The application of thin layers of matrix has special advantages, particularly when very high sensitivity is needed. Methods are also disclosed for the preparation of cellulose membranes precoated with a thin matrix layer for the direct deposition and analysis of aqueous samples. This technique circumvents the problems of mixing and dilution of samples when post addition of matrix is done and effectively allows small (nanoliter) volumes of samples to be applied to the target.

One aspect of the invention is the use of MALDI MS/MS techniques for the imaging of specimens, e.g. tissue sections, where the spatial arrangement of nucleic acid or polypeptide expression is to be determined. Two different approaches may be used: direct targeting of the tissue itself, or analysis of blotted targets previously exposed to the tissue.

The ability to image a specimen in order to obtain the detailed spatial arrangement of compounds in an ordered target specimen such as a slice of tissue using MALDI MS/MS is of enormous value in biological research. Tag daughter ion maps derived from contacting such specimens with tagged probes could provide for example details of drug modes of action, off-target activities of drugs, the roles of genes in developmental biology and mechanisms of disease.

Another aspect of the invention provides probe molecules suitable for the analysis of a specimen, such as a tissue sample, by MS particularly MALDI MS/MS-based imaging.

A further aspect of this invention provides methods for employing said probe molecules to image a specimen using MS (such as MALDI MS/MS) instrumentation.

A further aspect of this invention provides methods to allow for rapid acquisition of high-resolution images of a specimen by MS/MS.

MALDI scanning may be performed with an ultraviolet laser or an infrared laser.

Suitable matrices for UV MALDI include cinnamic acids, sinnapinic acid, hydroxypicolinic acid, nicotinic acid, hydroxybenzoic acid. Matrices for Infrared MALDI include glycerol, urea, succinic acid and water.

MS/MS analysis may be performed in, for example, an ion trap, TOF-TOF, quadrupole-TOF, ion-trap TOF, or Fourier transform ion cyclotron resonance instrument.

A still further aspect of the present invention provides improved techniques which use tandem mass spectrometry to determine, and preferably to visually depict, quantitative information regarding molecules of interest as a function of the spatial arrangement of numerous successive laser spots on a specimen.

A related aspect of the present invention improves the capability of MALDI MS/MS by providing improved methods for quantification of tag ions and for normalisation of the multiple spectra that are used to generate graphic displays of tag ions and consequently the distribution of the molecules of interest that are bound by the corresponding probes from which the tags are released.

A further aspect of the invention improves the speed of imaging mass spectrometry by reducing redundancy in image acquisition thus increasing the throughput of imaging mass spectrometry systems.

It is a further aspect of the present invention that improved techniques are provided for analysing the spatial arrangement of specific molecules within a specimen, such as a tissue sample, by tandem mass spectrometry analysis. A very thin sample layer may be generated and combined with an energy absorbent matrix to form a test specimen, which is then sequentially struck by a laser beam. The sample layer generated is preferably less than 50 microns in thickness.

One feature of the invention is that the intensity or normalised intensity of daughter ion fragments from tag ions may be graphically depicted as a function of the linear distance between successive laser spots.

Another feature of the invention is that the intensity of daughter ion fragments from tag ions may be normalised by comparison with the intensity of a calibrant ion.

It is a further feature of the invention that blotting techniques may be used to blot the sample on a blotting surface. The blotting surface may be a liquid absorbing surface, a chemically prepared surface, or a biologically prepared surface.

Another feature of the present invention is a technique for substantially drying the sample to minimise movement of sample molecules within the sample layer prior to striking the test specimen with laser pulses. The sample layer may be dried in a vacuum dessicator or a hydrolyser for at least two hours.

As used herein, the term “nucleic acid or polypeptide expression” refers to the expression of a variety of molecular species. Nucleic acids include messenger RNA (mRNA), micro-RNA (miRNA), transfer RNA (tRNA) and other endogenously expressed ribonucleic acids or deoxyribosenucleic acids. Similarly, polypeptide expression includes polypeptides, peptides, amino acids, peptide hormones, lipoproteins, carbohydrate-modified proteins, carbohydrates and other products of protein expression.

As used herein, the term “collision” where used on its own encompasses the tem “collision induced dissociation” (CID).

Particular non-limiting examples of the present invention will now be described with reference to the accompanying drawings, in which:

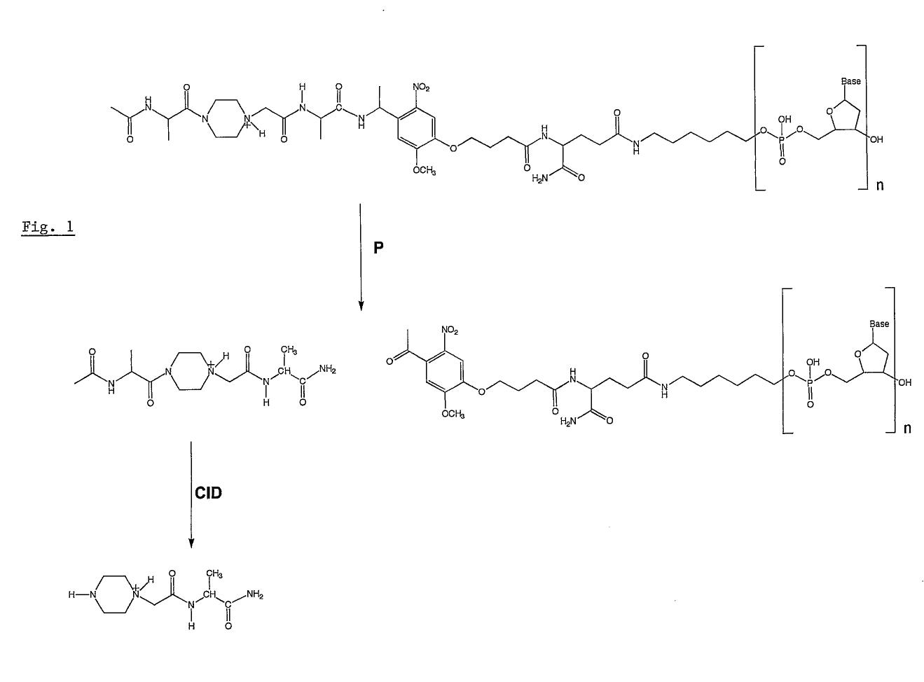

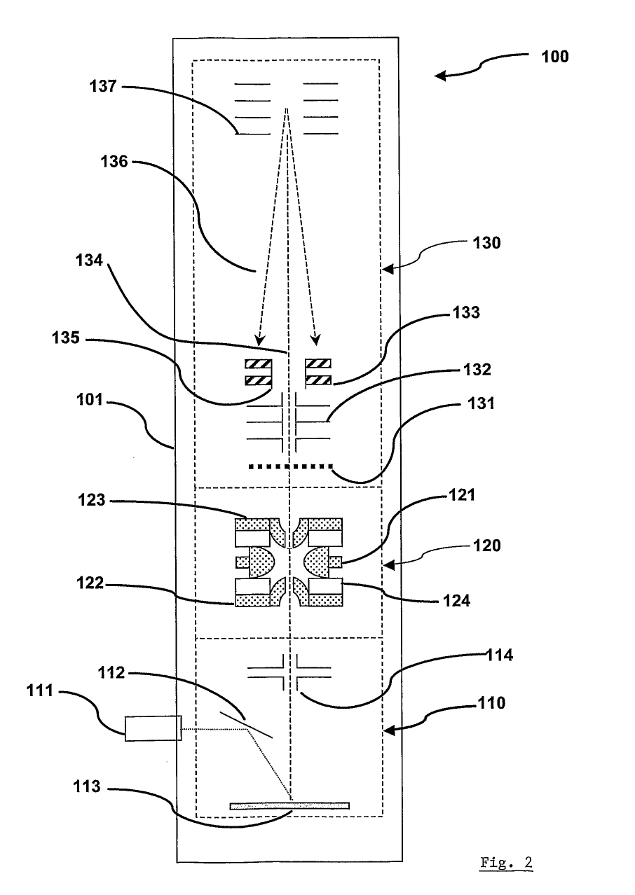

FIG. 1 depicts an example of a probe according this invention;

FIG. 2 is a schematic illustration of a tandem mass spectrometry apparatus for use in a method in accordance with the invention;

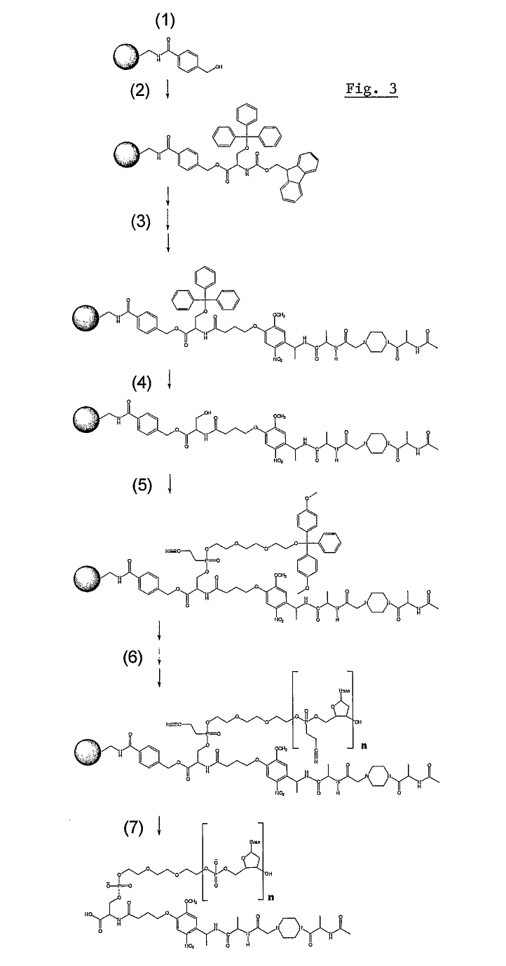

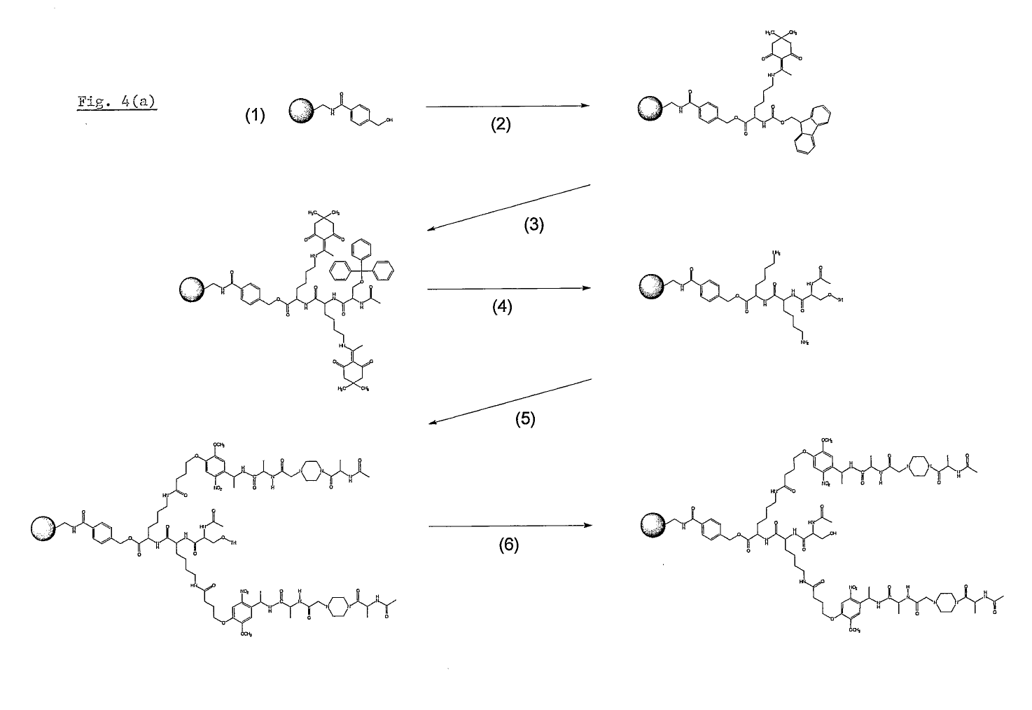

FIG. 3 is a schematic representation of a synthesis method for the preparation of singly labelled probes of this invention;

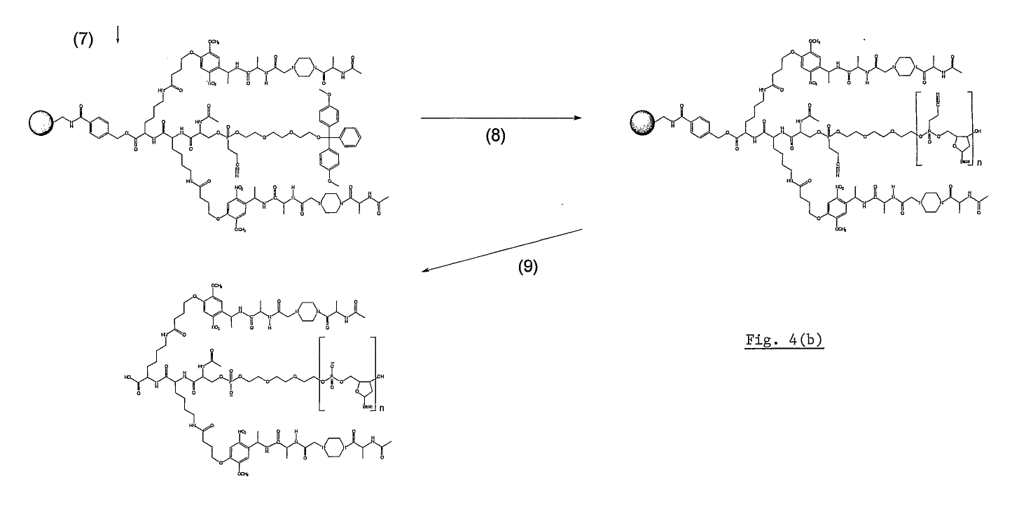

FIGS. 4(a) and 4(b) are a schematic representation of a synthesis method for the preparation of multiply labelled probes of this invention;

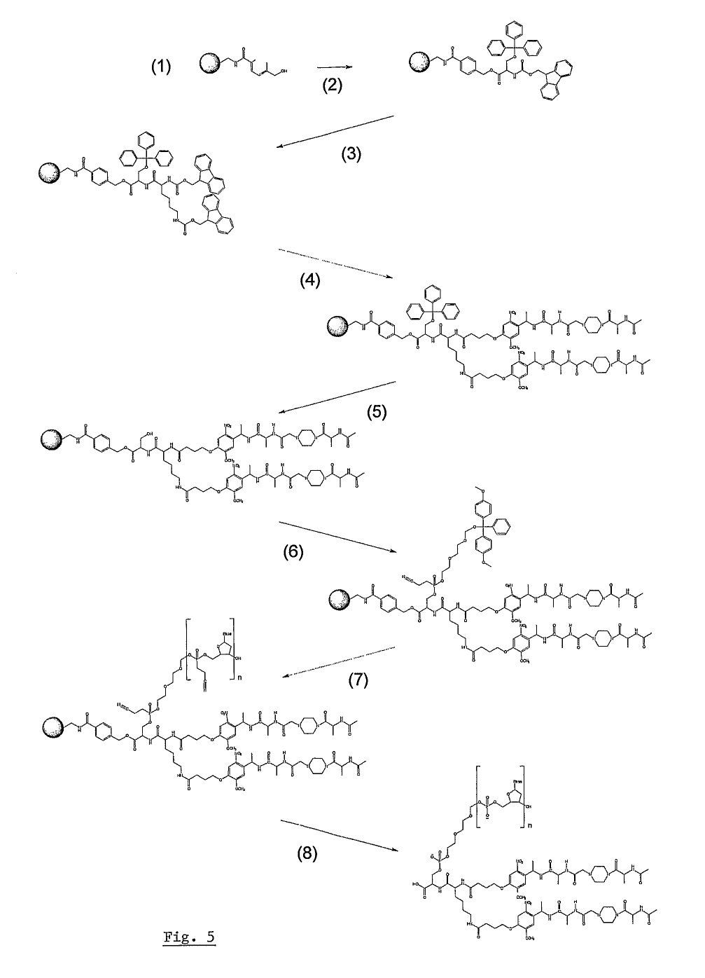

FIG. 5 is a schematic representation of an alternative synthesis method for the preparation of multiply labelled probes of this invention;

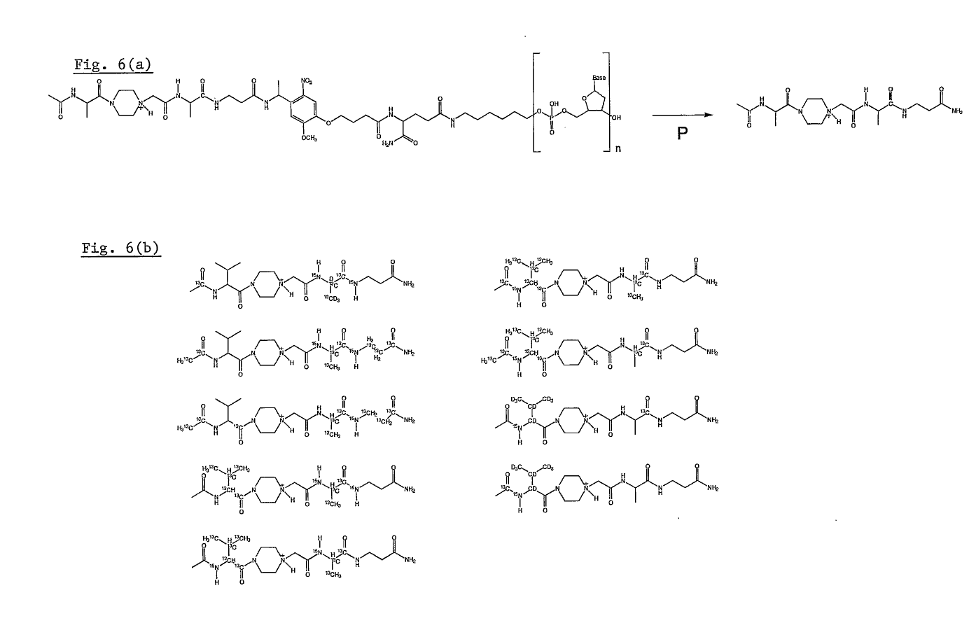

FIG. 6 illustrates a series of isobaric mass tag sets according to this invention;

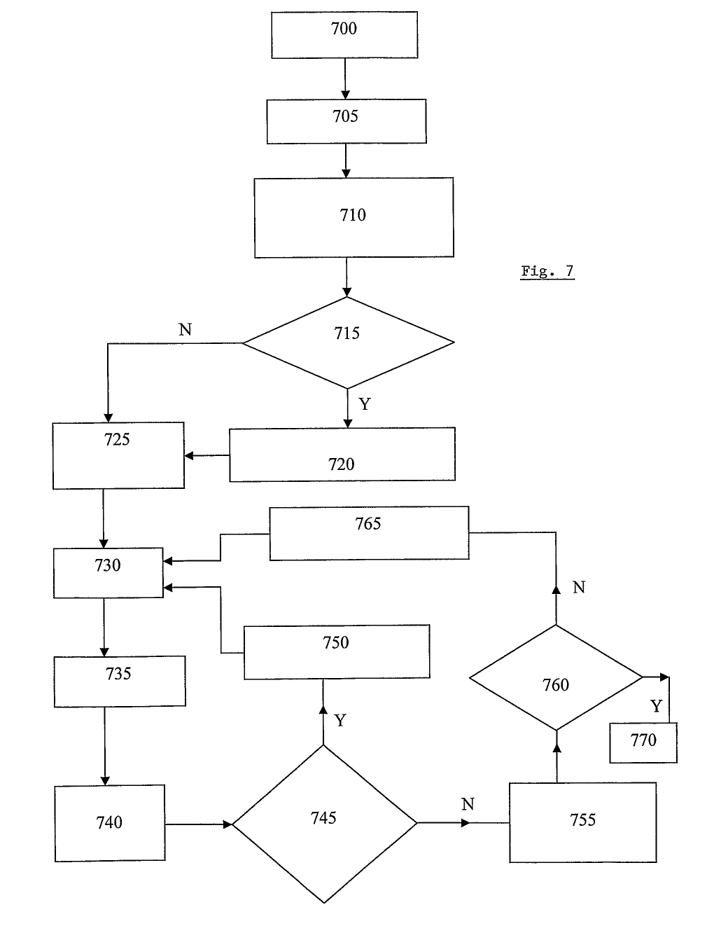

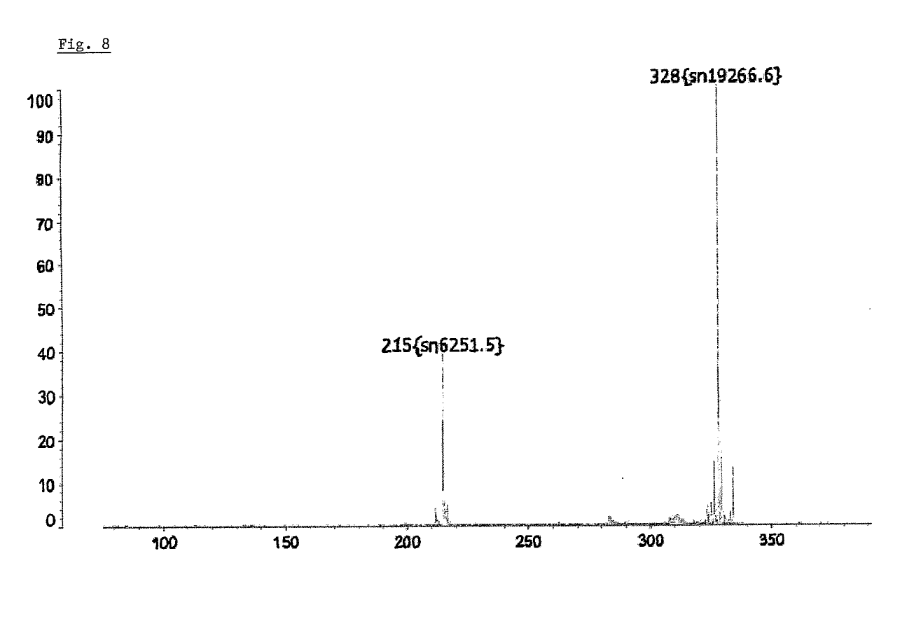

FIG. 7 is a flow diagram showing the method steps carried out in accordance with an embodiment of the invention;

FIG. 8 shows a mass spectrum of a photocleaved and desorbed tag peptide. The x-axis shows mass-to-charge ratio (m/z) and y-axis % intensity;

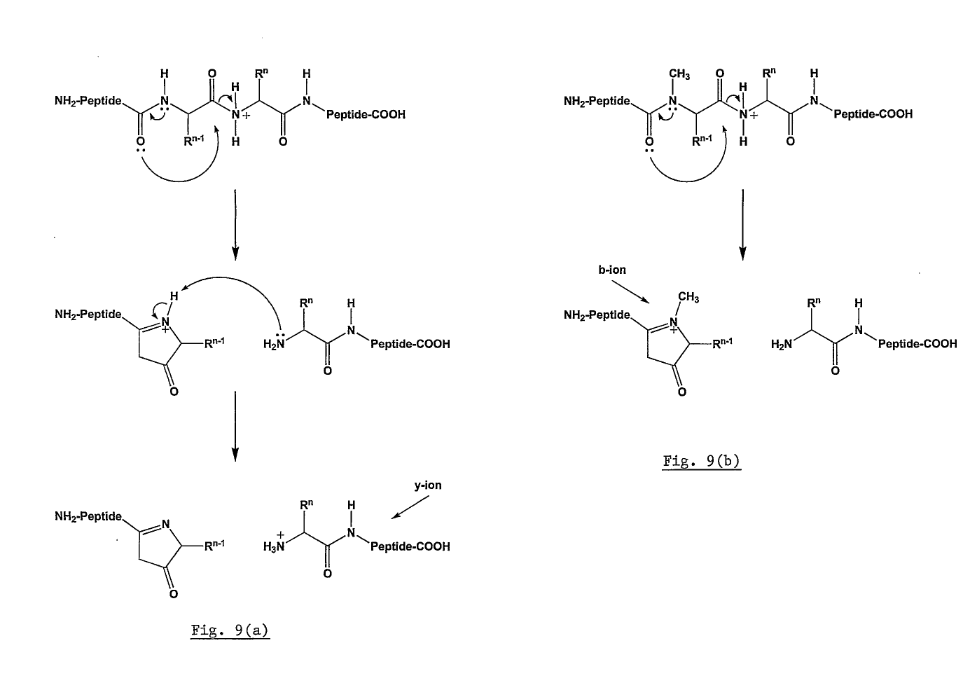

FIGS. 9a and 9b show alternative mechanisms for fragmentation of the amide backbone of a peptide;

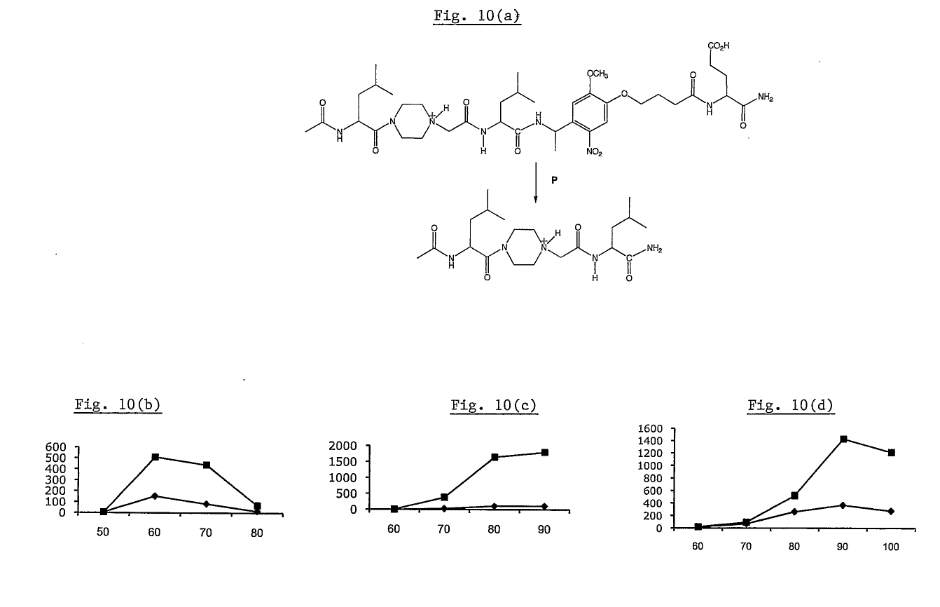

FIG. 10 shows (a) the structure of an exemplar mass tag Peptide Tag 1 (SEQ ID NO: 12; top) and its photocleavage daughter fragment (bottom), and (b-d) MALDI MS/MS spectra graphs following crystallisation of Peptide Tag 1 (SEQ ID NO: 12) in three different matrices (4-hydroxy-alpha-cyano-cinnamic acid [HCCA], 2,5-dihydroxybenzoic acid [DHB] and sinapinic acid [SA], respectively). In FIGS. 10(b)-(d), the x-axis shows laser power and the y-axis intensity, and -- indicates the peptide while -▪- indicates the fragment;

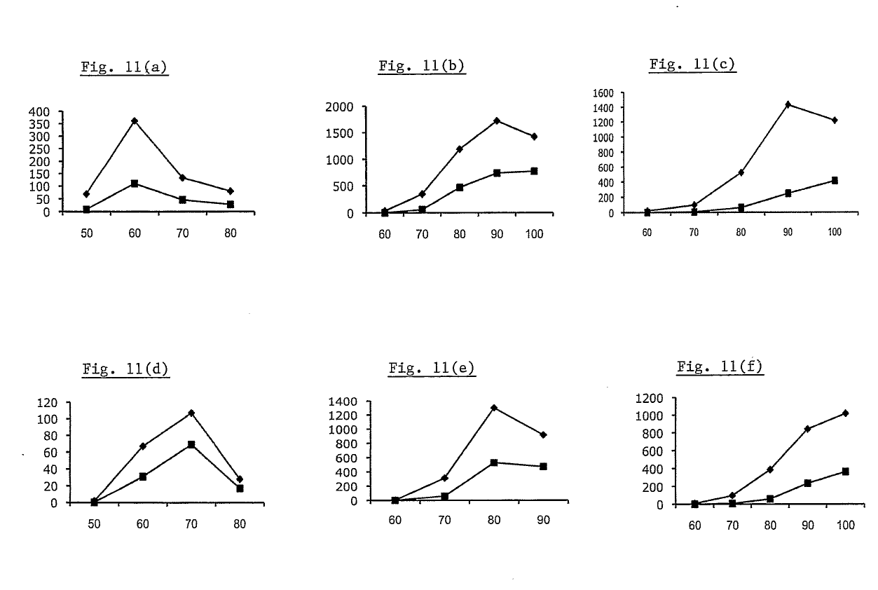

FIG. 11 shows further MALDI MS/MS spectra graphs of Peptide Tag 1 (SEQ ID NO: 12; -♦-) and its photocleavage daughter fragment (-▪-) using (a-c) Post Source Delay (PSD) and (d-f) Collision Induced Dissociation (CID) analysis. FIG. 11(a) and (d) show HCCA, FIG. 11(b) and (e) show DHB, and FIG. 11(c) and (f) show SA. In each graph, the x-axis shows laser power and the y-axis intensity;

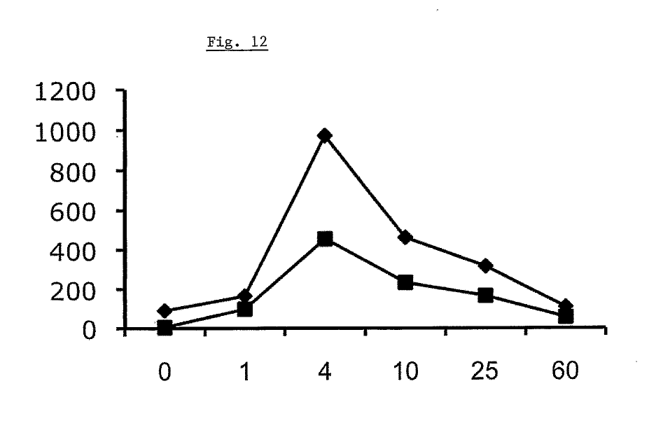

FIG. 12 is a graph showing optimal SA concentration for PSD MALDI MS/MS analysis of Peptide Tag 1 (SEQ ID NO: 12). The x-axis shows SA concentration in mg/ml, the y-axis intensity. Parent (-♦-) and daughter fragment (-▪-) results are shown;

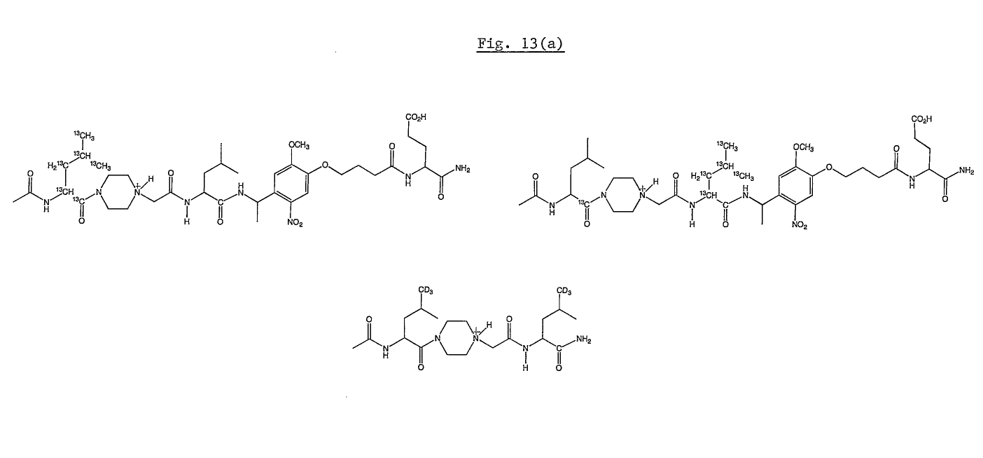

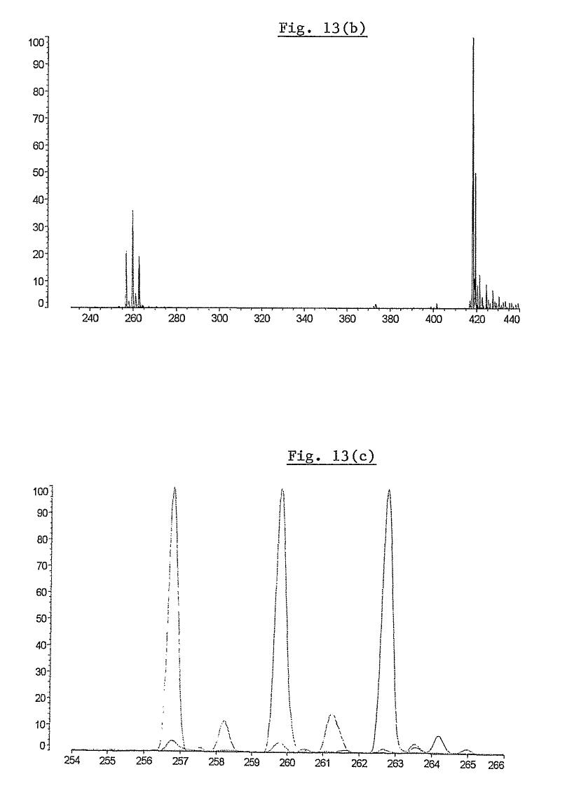

FIG. 13 shows (a) the structure of further exemplar mass tags Peptide Tag 2 (SEQ ID NO: 13; top left), Peptide Tag 3 (SEQ ID NO: 14; top right) and Peptide Tag 4 (SEQ ID NO: 15; bottom), (b) a MALDI MS/MS spectrum of desorbed and photocleaved oligonucleotide-conjugated Peptide Tag 2 (CONJUGATE2) and Peptide Tag 3 (CONJUGATE3), and (c) an intensity-adjusted spectra for the three tags. In FIG. 13(b) and (c), the x-axis shows mass-to-charge ratio (m/z) while the y-axis shows % intensity;

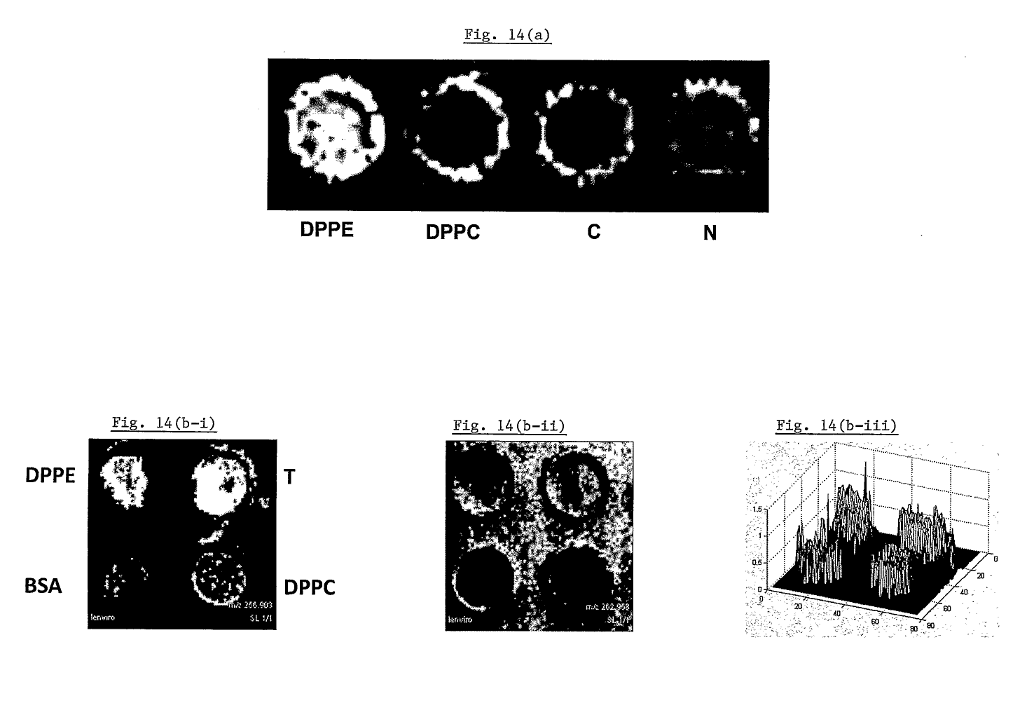

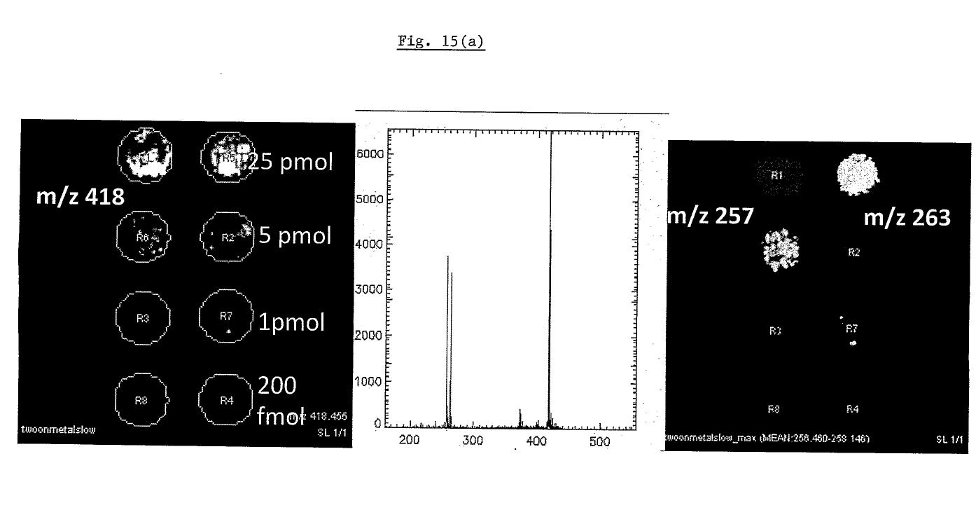

FIG. 14 shows (a) various MALDI MS/MS images detecting verapamil in the presence of different background ions (“DPPE”=dipalmitoyl phosphatidylethanolamine, “DPPC”=Dipalmitoyl Phosphatidylcholine, “C”=cholesterol, “N”=no lipid), MALDI MS/MS images of CONJUGATE2 (b-i; DPPE and DPPC as above, “BSA”=bovine serum albumin, “T”=tag alone) and CONJUGATE3 (b-ii; wells as b-i) in the presence of different backgrounds ions, and (b-iii) normalisation of the intensity of CONJUGATE2 with CONJUGATE3;

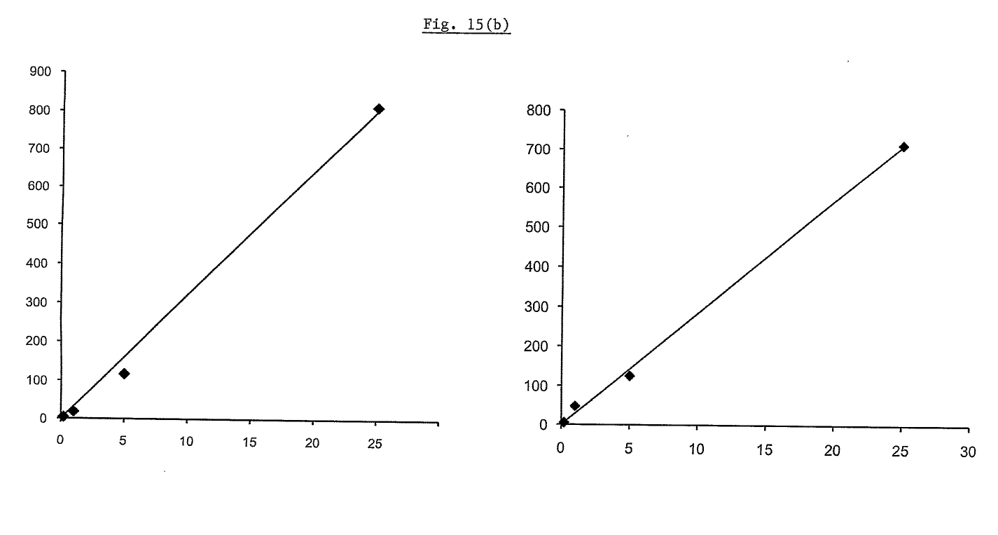

FIG. 15(a) and (b) shows results of a dilution series of CONJUGATE2 (left side) and CONJUGATE3 (right side), with FIG. 15(a) middle showing the intensities (y-axis) of the daughter ions at m/z (x-axis) 257 and 263. In each graph of FIG. 15(b), the x-axis shows pmol of probe molecule while the y-axis shows average intensity;

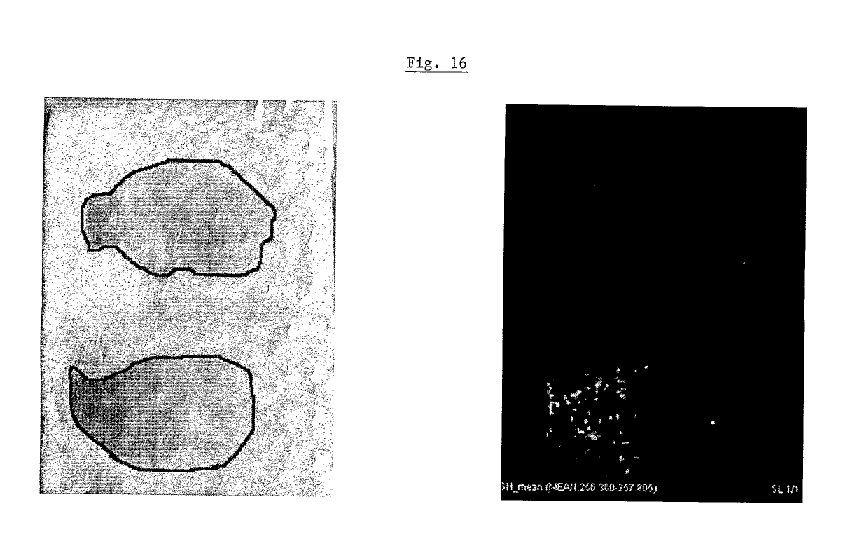

FIG. 16 shows images of a pair of mouse kidney tissue sections (left side) spiked with a synthetic Her2 target detected (right side) by MALDI MS/MS using CONJUGATE2;

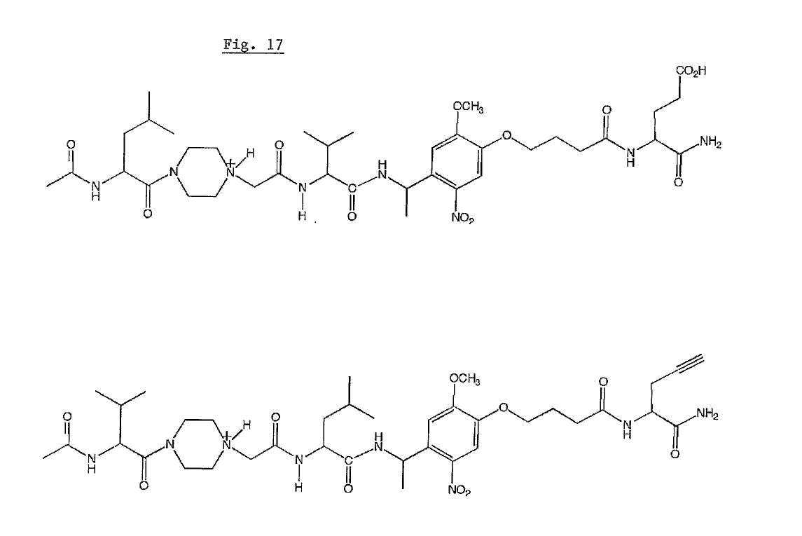

FIG. 17 shows the structure of two further peptide tags according to the present invention, viz. Peptide Tag 5 (SEQ ID NO: 19; top) and Peptide Tag 6 (SEQ ID NO: 20; bottom); and

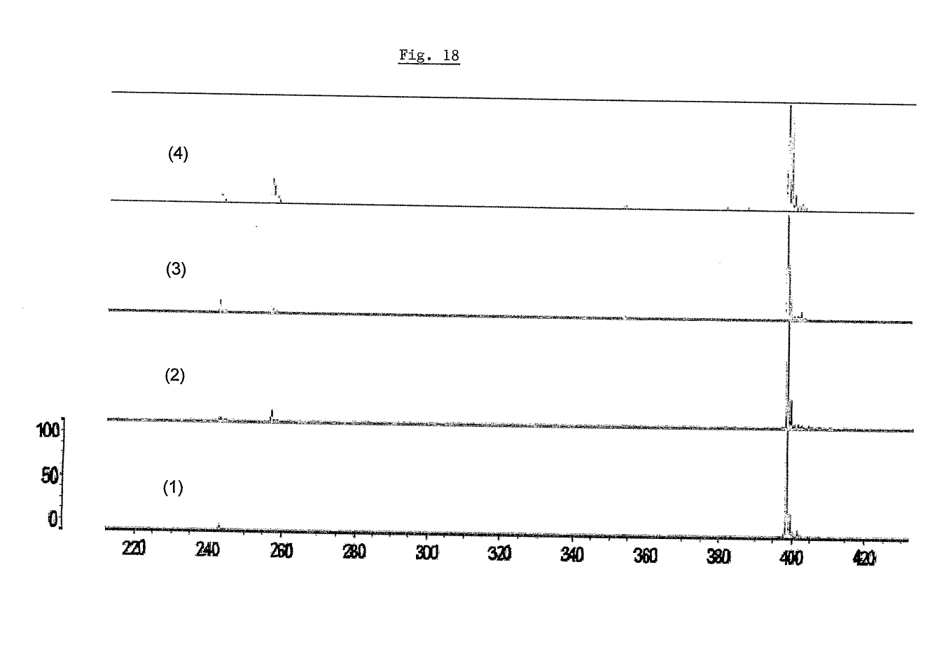

FIG. 18 shows a compound mass spectrum (x-axis in m/z; y-axis in intensity for each sub-spectrum) following photocleavage of the following four samples (1) CONGUGATE4 alone, (2) CONGUGATE4 with CONJUGATE_{—}1TAG, (3) CONGUGATE4 with CONJUGATE_{—}2TAG, and (4) CONGUGATE4 with CONJUGATE_{—}4TAG.

Imaging of gene and protein expression directly in tissues is a highly informative way to determine gene function and activity while relating the expression to physical features of the tissue. Typically, radiolabels, fluorescent labels or other optical labels are used, but all of these standard techniques are limited by the number of tags available for use with these techniques. The present invention allows for simultaneous detection of expression of many, if not all, genes in a sample. This allows for better use of precious samples, ensures that changes in one gene can be compared with others directly and ensures that all genes are subjected to the same experimental conditions, thus improving data quality.

Another issue with optical tags is that most tags are quite different chemically from each other, which introduces variation in labelling and detection efficiencies. These label biases can distort quantification of gene expression quite significantly. The present invention addresses this issue.

In one aspect of the invention, a probe molecule is provided comprising an oligonucleotide coupled to one or more mass tags by photocleavable linkers:

[mass tag-photocleavable linker]ₙ-oligonucleotide,
where “n” is any positive integer, for example n=1, 2, 3, 4, 5, 6, 7, 8, 9, 10 or higher integer.

In a further embodiment of this first aspect of the invention the mass tag has the form:

mass modifier 1-collision cleavable linker-mass modifier 2.

The mass tag additionally comprises at least one charge-carrying group. If more than one charge-carrying group is used, these may all be on one side of the collision cleavable linker. The charge-carrying group may be a separate component from the mass modifier or the mass modifier may itself comprise a charge-carrying group.

Alternatively the collision cleavable linker may comprise a charge-carrying group as long as the charge ends up consistently on only one of the fragments generated by collision.

The mass tag may comprise or consist of a peptide. For the purposes of analysis by MS/MS, the collision cleavable linker may comprise a group that is readily cleaved upon low energy collision in the mass spectrometer. The collision sensitive group may be selected from a proline linkage, a piperazine linkage, a piperidine linkage, an aspartic acid linkage or a linkage comprising aspartic acid and proline adjacent to each other with the aspartic acid on the N-terminal side of the proline residue, as is described below in more detail.

A set of two or more oligonucleotides conjugated to mass tags by photocleavable linkers may be provided where the mass tags are isobaric but may be resolved from each other by tandem mass spectrometric methods. Isobaric means that the total mass of each mass tag is the same for every tag in a set. This is achieved by ensuring that the sum of the masses of mass modifier 1 and mass modifier 2 are the same for each mass tag in the set but the actual masses of mass modifier 1 and mass modifier 2 are different from each other. Tags are resolved by fragmentation of the collision cleavable linker according to the methods of the invention to release tag fragments that have uniquely resolvable masses.

The isobaric mass tags may be isotopes of each other. This may be achieved by synthesising peptides that have the same sequence where mass modifier 1 and mass modifier 2 comprise amino acids that have been mass modified using isotopes, preferably stable isotopes. Preferred stable isotopes include ¹³C, ¹⁵N, ¹⁷O, ¹⁸O, ³⁴S and deuterium (²H).

The tags of this invention can be produced in large numbers and they can be constructed as isotopic sets so that their properties are as similar to each as can possibly be achieved. Thus, imaging of tissue sections, microarrays or other planar samples with the tags of this invention can offer significant advantages over the prior art.

In a typical imaging experiment labelled probes are contacted with the target specimens, for example a tissue or array. Probes that find their target will bind, allowing unbound probes to be removed, typically by washing or rinsing the sample with a suitable buffer. Binding buffers and washing buffers typically contain only volatile salts such as ammonium carbonate, ammonium acetate and similar compounds. After removal of unbound probes the planar sample is typically coated with a MALDI matrix material or laid on a surface coated with a MALDI matrix. The planar sample may then be introduced into a suitable laser desorption mass spectrometer where it is scanned. A raster scan may be performed or a directed analysis of specific locations may be carried out. In a raster scan, the laser is focussed successively on adjacent locations in a line and the whole planar sample is scanned line-by-line so that the whole sample is imaged. At each location a mass spectrum is obtained. In the context of this invention, the mass spectrum is preferably an MS/MS spectrum. The desorbed ions from each location enter the mass analyser. Ions with the mass-to-charge ratio that corresponds to ratios expected for tags ion are gated into a collision cell (or in the case of ion traps and FT-ICR instruments the ions are selectively retained in the trap). In the collision cell or trap, the ions are subjected to collision with a bath gas such as argon or helium or neon. The fragments are then analysed to detect the characteristic daughter ions that are produced by the tags of this invention. This process of selection and detection enhances selectivity and specificity enabling tags to be distinguished from other ions in the sample. This is an important feature of this invention as tissues comprise complex mixtures of molecules that will generate complex mass spectra. The intensities of the tag daughter ions may be recorded for each location that is analysed on the planar sample.

FIG. 1 illustrates an example of a labelled probe molecule of the form “[mass tag-photocleavable linker]₁-oligonucleotide” in accordance with the invention, where the mass tag is a peptide. As a result of the scan, an ionised mass tag (with [M+H]⁺=328.2 in this example) is photocleaved (shown as “P” in FIG. 1) from the probe molecule. Then, in the collision cell or trap, the mass tag is fragmented by collision with a bath gas (a process known as collision induced dissociation [CID]) to produce a fragment (in this example with [M+H]⁺=215.15). It is these resulting fragments that are analysed to provide an indication of the amounts target molecules in different portions of the specimen.

A map or image of the relative amounts of the target molecule identified by each tag can then be constructed. Each tag can be displayed in a different colour on a single image or each tag can be viewed as a distinct image.

The area or spot irradiated by the desorption laser can be varied on some instruments, and the spot area is an important variable in the imaging process particularly for tissue imaging. The larger the spot size, the fewer spots needed to cover a given area and the lower the amount of data acquired, thereby reducing data burden, and scanning time. However, the larger the spot size, the poorer the ability to spatially resolve adjacent locations of molecules of interest. With very small spot sizes, i.e. 1 μm diameter or less, the resolution of the resulting image improves, but this is at the expense of time (more spots needed to cover a given area), and at the expense of increased data load. Varying spot size can be used as a way to gather data quickly before focussing on an area of interest. For example, the sample may be exposed to a small number of larger spot sizes initially to provide a “survey scan”. If the molecules of interest are present at specific location, then smaller spot sizes can be used to increase the resolution of the image so produced.

The tissue sections may be dried before analysis, such as in a vacuum desiccator. Alternatively, tissue sections may be fixed, for example using paraformaldehyde.

In the context of tissue sections, the flatness of the sample is not critical to the methods of this invention but most tissue sections will start flat during the sectioning process. Thus, to ensure the resulting image matches the original structure of the tissue, it may be important to maintain the flatness of section during the many preparation steps that are needed prior to analysis. For thick sections (80-100 μm), it may be necessary to dry the section slowly over several days to get best results as fast drying tends to warp the specimen making it complicated to mount. Thin sections (1-10 μm) dry much faster and can be kept flat on the mounting membrane more easily. Surface levels that differ significantly in height can produce different mass shifts in simple Time-of-Flight analysis, although with Trap-TOF geometries this is not a major issue as the ions are collected in the trap and subjected to collisional cooling prior to further analysis.

The invention can be practiced with various different kinds of mass spectrometer and some of the more favourable geometries are described below.

Mass spectrometers typically comprise the following components:

### Interface-Mass Analyser-Detector-Data Capture and Analysis System.

For the purposes of Imaging Mass Spectrometry, it is preferred that Laser Desorption Interfaces are used. Typically these require that the sample is introduced into a chamber in the instrument, which is evacuated and where the sample can be exposed to a laser beam. The laser source typically generates a laser beam that passes through a beam adjusting mechanism or mask and/or suitable optics that then strikes a sample target containing the test sample, thereby releasing ions of interest with various mass-to-charge ratios, which are then analysed by the mass analyser. Ion optics deliver the ions to the mass analyser. Most MALDI instruments have a capability to focus the laser at successive locations on a planar sample to allow a sample to be scanned. Atmospheric Pressure Laser Desorption interfaces are also commercially available and are suitable for the practice of this invention (Schneider et al., 2005, J. Am. Soc. Mass Spectrom 16: 176-182).

Most mass analysers separate ions according to mass-to-charge ratios. They may also isolate or gate ions with specific mass-to-charge ratios and subsequently fragment these selected ions. Ions, whether trapped, gated and/or fragmented are then delivered to a detector, which counts the ions. These counts are then converted into mass spectra by the data capture and analysis system.

Fourier Transform instruments detect radio waves that are emitted by moving ions trapped in the instrument. These instruments are operated so that the frequencies of the radio waves can be used to determine the mass-to-charge ratios of trapped ions. In these systems ions are not detected directly but most of the same principles apply in the subsequent data analysis as the signals are typically converted into ion counts.

### Imaging by MALDI MS/MS Mass Spectrometry

MALDI mass spectrometry has become a widely used tool for the analysis of many types of biological molecules, especially peptides and proteins. MALDI requires that the biomolecule solution be embedded in a large molar excess of a photo-excitable “matrix”. The application of laser light of the appropriate frequency results in the excitation of the matrix, which in turn leads to rapid evaporation of the matrix along with its entrapped biomolecule. Proton transfer from the acidic matrix to the biomolecule gives rise to protonated forms of the biomolecule, which can be detected by positive ion mass spectrometry, particularly by Time-Of-Flight (TOF) mass spectrometry. Negative ion mass spectrometry is also possible by MALDI TOF. MALDI imparts a significant quantity of translational energy to ions, but tends not to induce excessive fragmentation despite this. Accelerating voltages can again be used to control fragmentation with this technique though. This technique is highly favoured for the determination of peptide mass fingerprints due to its large mass range, due to the prevalence of singly charged ions in its spectra and due to the ability to analyse multiple peptides simultaneously.

The photo-excitable matrix comprises a “dye”, i.e. a compound that strongly absorbs light of a particular frequency, and which preferably does not radiate that energy by fluorescence or phosphorescence but rather dissipates the energy thermally, i.e. through vibrational modes. It is the vibration of the matrix caused by laser excitation that results in rapid sublimation of the dye, which simultaneously takes the embedded analyte into the gas phase.

### Mass Analysers

### Time-Of-Flight Mass Analysers

As the name implies, Time-of-flight mass analysers measure the time it takes for ions to travel a predetermined distance under the influence of a predetermined potential difference. The time-of-flight measurement allows the mass-to-charge ratio of ions striking a detector to be calculated. These instruments measure the arrival of almost all of the ions in a sample and as a result can be quite sensitive, although selectivity with this technique is more difficult to achieve. This technique can also detect ions with higher mass-to-charge ratios than can be typically measured in an ion trap or quadrupole mass spectrometer. TOF mass analysers are presently widely used with MALDI. Hybrid instruments that enable MS/MS analysis using TOF analysers are particularly well suited to the methods of this invention, including quadrupole-TOF geometries, trap-TOF geometries and TOF-TOF geometries.

FIG. 2 show an embodiment of an instrument 100 suitable for imaging mass spectrometry in accordance with the methods of this invention. The MALDI MS/MS instrument, 100, comprises a vacuum housing, 101, that contains the instrument components and which has a pumping device and other standard vacuum components (not shown).

The instrument 100 comprises a laser desorption ionisation (LDI) source 110. This is shown contained within the vacuum housing 101 although atmospheric pressure LDI sources are also applicable with this invention. The instrument 100 further comprises a quadrupole ion trap 120 and a Time-Of-Flight analyser 130.

The LDI source 110 comprises a laser 111, optionally a mirror 112 and optionally other optical elements (not shown). The mirror may be movable by servo motors or other means to allow the laser (dotted line leaving the laser 111) to be scanned across the sample stage 113, where planar samples for analysis are deposited. The ions that exit the LDI source 110 may be steered into a quadrupole ion trap 120 by electrostatic focusing elements 114. The quadrupole ion trap 120 traps ions created within the LDI source 110. The quadrupole ion trap 120 typically includes a ring electrode 121 that is separated from a pair of endcap electrodes 122 and 123 by dielectric material 124. The elements 114 and endcap 122 may function as an einsel lens. Typically, the first and last element of the einsel lens (the first element of 114 and electrode 122) can be biased at the same voltage, such as ground potential, and the middle element (the second element of 114) is biased at a different potential. Alternatively, the lenses can have progressively decreasing potential to accelerate the ions. Other focusing elements have been tested and may include multipole ion guides, such as quadrupole, hexapole, or octapole ion guides.

The ions enter the quadrupole ion trap 120 and are stabilised and stored within the trap by the application of an alternating current to the ring electrode 121 in a manner known in the art. The endcaps 122 and 123 are usually held at a constant voltage, such as ground potential, however, auxiliary oscillating current may be applied. The range of ion masses that are stored efficiently depends on the frequency and amplitude of the current applied to the ring electrode, it is typically of radio frequency (such as 1 MHz) and a few hundred to a few thousand volts peak to peak, but can have other values. The ions may continuously accumulate within the trap 120. Waveforms can be applied to one or both endcap electrodes 122 and 123 and/or to the ring electrode 121 to excite specific ion masses in the trap 120 in order to eject them from stable orbits, to prevent them from accumulating, or to excite them to more energetic orbits to cause them to dissociate with background gas in order to produce fragment ions of the selected ions. Each ion mass has a distinct resonance condition. Many different ion masses may be excited simultaneously by applying a superposition of many frequencies. The frequency spectrum may be generated by a variety of prior art methods. In this embodiment, the arbitrary waveform is formed by superimposing the sum of individual periodic waveforms corresponding to the frequency and amplitude most suited for exciting each ion mass to the desired effect. In one embodiment this waveform may be applied to the exit endcap 123, although it is to be understood that effective excitation may be achieved by application of the waveform to other electrodes in the ion trap.

Following accumulation of ions and the optional manipulation of ions consisting of selective ion ejection and selective collision-induced ion dissociation, the remaining ions in the quadrupole ion trap 120 are mass analysed by ejecting all the ions into a Time-Of-Flight mass analyser 130. Ion ejection to the mass analyser 130 may be accomplished by applying a high voltage pulse to the ion trap exit endcap 123. Alternatively, a high voltage pulse may be applied to the entrance endcap 122 to “push” the ions into the detector 130, or two oppositely-phased pulses may be applied to both endcaps 122 and 123 in a “push-pull” manner to extract ions into the TOF mass analyser 130.

The extracted ions can be accelerated to a higher energy by an acceleration grid 131. The accelerated ion pulse may be focused and collimated by a electrostatic lens assembly 132. This is shown as a three-element einsel lens, however other configurations may be used, such as a two-element assembly. The third element in the einsel lens configuration can make use of the back plate of the detector 133.

The accelerated and collimated ion packet passes through a hole 134 in the coaxial detector 133. A cylinder 135 may be provided in the detector hole 134 to keep a uniform voltage potential for the traversing ions and is electrically isolated from the detector plates themselves (described below). The ions travel through a drift tube 136 under field-free conditions where ions of different mass travel at different speeds and spread out in space. The ions may then reach a reflectron section 137 of the mass detector where they are reversed in direction. This operation acts to focus ions of different initial kinetic energies. The ions then travel back toward the front of the detector 133 where they impact and are recorded as a signal. The resulting signal from the detector is measured with electronics that can distinguish the different arrival times of different ion masses. Although a reflectron 137 is shown with a coaxial detector in the mass spectrometer (100), it is to be understood that the reflectron 137 may also be of an off-axis design, or the TOF mass analyser 130 may be of a linear design with the detector plate 133 at the end of the drift tube 136.

The instrument 100 is very well suited for the practice of this invention. A tissue section or other planar sample can be scanned by a laser in the ion source 110. Tag ions of the present invention can then be collected in the quadrupole ion trap 120. Non-tag ions, i.e. ions with different mass-to-charge ratios from the tags can be selectively ejected from the trap by application of appropriate waveforms to the trap electrodes. The tag ions can then be fragmented in the trap by application of suitable waveforms. Then, by applying appropriate potentials to the electrodes 122 and 123, fragment ions can be pushed and/or pulled into the TOF analyser 130 for mass determination and detection.

In the TOF-TOF geometry, the ion source is linked to a TOF drift tube in which ions can separate according to flight time. This drift region is separated from a full TOF analyser by a gate and collision cell arrangement that allows ions from the first drift region to be selected and fragmented prior to mass spectrometry analysis in the second TOF analyser where the mass-to-charge ratios of the gated and fragmented ions can be determined. When the gate and collision region are not in use the instrument acts as a single MS-mode TOF analyser. This geometry is quite advantageous for the purposes of this invention due to its high repetition rate, i.e. the rate at which spectra can be obtained.

### Ion Traps

Ion Trap mass analysers are related to the quadrupole mass analysers. An ion trap generally has a 3 electrode construction—a cylindrical electrode with “cap” electrodes at each end forming a cavity. A sinusoidal radio frequency potential is applied to the cylindrical electrode while the cap electrodes are biased with DC or AC potentials. Ions injected into the cavity are constrained to a stable circular trajectory by the oscillating electric field of the cylindrical electrode. However, for a given amplitude of the oscillating potential, certain ions will have an unstable trajectory and will be ejected from the trap. A sample of ions injected into the trap can be sequentially ejected from the trap according to their mass/charge ratio by altering the oscillating radio frequency potential. The ejected ions can then be detected allowing a mass spectrum to be produced.

Ion traps are generally operated with a small quantity of a “bath gas”, such as helium, present in the ion trap cavity. This increases both the resolution and the sensitivity of the device as the ions entering the trap are essentially cooled to the ambient temperature of the bath gas through collision with the bath gas. Collisions both increase ionisation when a sample is introduced into the trap and dampen the amplitude and velocity of ion trajectories keeping them nearer the centre of the trap. This means that when the oscillating potential is changed, ions whose trajectories become unstable gain energy more rapidly, relative to the damped circulating ions and exit the trap in a tighter bunch giving narrower larger peaks.

Ion traps can mimic tandem mass spectrometer geometries, in fact they can mimic multiple mass spectrometer geometries allowing complex analyses of trapped ions. A single mass species from a sample can be retained in a trap, i.e. all other species can be ejected and then the retained species can be carefully excited by super-imposing a second oscillating frequency on the first. The excited ions will then collide with the bath gas and will fragment if sufficiently excited. The fragments can then be analysed further. It is possible to retain a fragment ion for further analysis by ejecting other ions and then exciting the fragment ion to fragment. This process can be repeated for as long as sufficient sample exists to permit further analysis. It should be noted that these instruments generally retain a high proportion of fragment ions after induced fragmentation. These instruments and FTICR mass spectrometers (described below) represent a form of temporally resolved tandem mass spectrometry rather than spatially resolved tandem mass spectrometry which is found in linear mass spectrometers.

Traps can also be combined with TOFs in so-called TRAP-TOF geometries. These are quite advantageous for the purposes of this invention as the TRAP collects ions, selects ions and fragments ions that are then injected into the TOF for mass determination. The TOF typically has much higher mass resolution than traps and a wider dynamic range. In addition, the trap can continue isolating ions while the TOF is analysing ions giving good duty cycle.

### Fourier Transform Ion Cyclotron Resonance Mass Spectrometry (FTICR MS)

FTICR mass spectrometry has similar features to ion traps in that a sample of ions is retained within a cavity but in FTICR MS the ions are trapped in a high vacuum chamber by crossed electric and magnetic fields. The electric field is generated by a pair of plate electrodes that form two sides of a box. The box is contained in the field of a superconducting magnet which in conjunction with the two plates, the trapping plates, constrain injected ions to a circular trajectory between the trapping plates, perpendicular to the applied magnetic field. The ions are excited to larger orbits by applying a radio-frequency pulse to two ‘transmitter plates’, which form two further opposing sides of the box. The cycloidal motion of the ions generates corresponding electric fields in the remaining two opposing sides of the box, which comprise the ‘receiver plates’. The excitation pulses excite ions to larger orbits which decay as the coherent motions of the ions is lost through collisions. The corresponding signals detected by the receiver plates are converted to a mass spectrum by Fourier Transform (FT) analysis.

For induced fragmentation techniques these instruments operate in a similar manner to an ion trap: all ions except a single species of interest can be ejected from the trap. A collision gas can be introduced into the trap and fragmentation can be induced. The fragment ions can be subsequently analysed. Generally fragmentation products and bath gas combine to give poor resolution if analysed by FT analysis of signals detected by the ‘receiver plates’, however the fragment ions can be ejected from the cavity and analysed in a tandem configuration with a quadrupole, for example.

### Reagents

The methods of this invention require a variety of reagents, which are described in detail below.

### Probe Synthesis

Isobarically mass tagged probe oligonucleotides and any other oligonucleotides, such as multimeric oligonucleotides (for example branched or comb oligonucleotides; described below) and the components of dendrimers can be synthesised using standard oligonucleotide synthesis methods known in the art. Preferred methods are purely synthetic methods, for example, by the cyanoethyl phosphoramidite method (Beaucage & Caruthers, 1981, Tetrahedron Lett. 22: 1859-1862; McBride & Caruthers, 1983, Tetrahedron Lett. 24: 245-248). Synthetic methods useful for making oligonucleotides are also described by Ikuta et al. (1984, Ann. Rev. Biochem. 53: 323-356), (phosphotriester and phosphite-triester methods), and Narang et al. (1980, Methods Enzymol. 65: 610-620) (phosphotriester method). PNA molecules can be made using known methods such as those described by Nielsen et al. (1994, Bioconjug. Chem. 5: 3-7). PNA is a preferred oligonucleotide analogue for the practice of this invention as PNA is able to hybridise under low salt conditions, even in water if necessary. This means that hybridisation to nucleic acids in tissue sections can take place under whatever conditions suit the experiment.

For many applications of the oligonucleotides of the present invention it is useful to know how stable they are, or more specifically at what temperature they will dissociate. The stability of DNA duplexes can be calculated using known methods for prediction of melting temperatures (Breslauer et al., 1986, PNASUSA 83(11): 3746-3750; Lesnick & Freier, 1995, Biochemistry 34:10807-10815; McGraw et al., 1990, Biotechniques 8: 674-678; and Rychlik et al., 1990, Nucleic Acids Res. 18: 6409-6412).

### Nucleic Acid Analogues

In embodiments of the invention, it may be useful to employ nucleic acid analogues with enhanced binding affinity compared to natural phosphodiester deoxyribosenucleic acids. It is known that RNA analogues with certain modifications at the 2′ position of the ribose ring show enhanced binding affinity for RNA targets compared to corresponding DNA/RNA hybrids (see Cummins et al., 1995, Nucleic Acids Res. 23(11): 2019-24). These RNA analogues also show reduced binding affinity for DNA compared to DNA/DNA hybrids (Tsourkas et al., 2003, Nucleic Acids Res. 31(6): 5168-74). The ability to bind preferentially to RNA over DNA with enhanced melting temperature makes 2′-modified analogues particularly useful for in situ hybridisation applications for detection of alternatively spliced RNA in a background of genomic DNA.

2′-O-methyl analogues in particular are readily available as phosphoramidite monomers for automated synthesis and are suitable for use with this invention. Additionally or alternatively, 2′-fluoro-modified analogues may be used.

Another nucleic acid analogue for use with this invention are “bridged” analogues such as locked nucleic acids (“LNA”; Thomsen et al., 2005, RNA. 11(11):1745-8) and 2′-4′-BNA(NC) (Rahman et al., 2008, J Am Chem. Soc. 130(14): 4886-96). Bridged nucleic acid analogues show enhanced binding affinity for RNA compared with their natural nucleic acid counterparts, and are thus suitable for in situ hybridisation applications. Bridged analogues also show enhanced binding affinity for DNA compared with their natural nucleic acid counterparts, and are therefore useful for detection of chromosomal targets such as chromosomal translocations and for the detection of labelled cDNAs.

It is not normally desirable to synthesise probes that are comprised entirely of bridged analogues. Hence LNA monomers are typically introduced every third base into DNA probes (Válóczi et al., 2004, Nucleic Acids Res. 32(22): e175; Obernosterer et al., 2007, Nat. Protoc. 2(6): 1508-14). LNA monomers may be introduced into 2′-O-methyl oligonucleotide sequences to enhance binding affinity of the resultant probe (Kierzek et al., 2005, Nucleic Acids Res. 33(16): 5082-93). When LNA monomers are introduced into 2′-O-methyl probes, the LNA monomers may be positioned every second base but in one embodiment not at the 5′ end of a probe.

PNA is another analogue for use with this invention (Nielsen et al., 1994, Bioconjug Chem. 5(1): 3-7). PNA has enhanced binding affinity for both DNA and RNA targets compared to DNA probes. PNA is less soluble than other DNA analogues and it is currently difficult to produce usable PNA oligonucleotides with a length greater than 20 bases. Hence PNA probes may be shorter than probes made with sugar/phosphate backbones. The invention encompasses chimeric probes comprising lengths of PNA and DNA (see Uhlmann, 1998, Biol. Chem. 379(8-9): 1045-52). These chimeric probes may be longer than PNA-only probes.

### Mass Tagged Oligonucleotides

A variety of mass tags can be used with this invention although preferred mass tags (also known as “tags” or “mass markers”) are disclosed in WO 97/27327, WO 97/27325, WO 97/27331 and WO 03/025576. Those publications disclose tags that comprise polyamide compounds, essentially peptides or peptide-like tags, which means that these tags can be prepared using a number of peptide synthesis methods that are well known in the art (see for example Jones, 1991, “The chemical synthesis of peptides”, Oxford University Press; Fields & Noble, 1990, Int J Pept Protein Res 35(3): 161-214; Albericio, 2000, Biopolymers 55(2):123-139). In addition, the use of peptide and peptide-like tags enables coupling of these tags to oligonucleotides using a variety of peptide conjugation techniques that are known in the art. Methods for coupling peptides to oligonucleotides “on column” via 5′ amine functionalities are disclosed in Zaramella et al. (2004, J Am Chem Soc 126(43): 14029-14035). Methods for conjugating peptides to oligonucleotides via thiol groups at the termini of the oligonucleotides are disclosed in Arar et al. (1995, Bioconjug Chem. 6(5): 573-577). Oligonucleotides can be coupled to peptides with terminal cysteine residues as disclosed in Wei et al. (1994, Bioconjug Chem. 5(5): 468-74).

In one approach, peptide tags can be synthesised on Controlled Pore Glass (CPG) beads of the kind used for DNA synthesis. As long as these peptides are suitably protected and as long as the peptide has a free hydroxyl (or trityl protected hydroxyl) at the end of the peptide synthesis, an oligonucleotide can be synthesised directly on the peptide (Haralambidis et al., 1990, Nucleic Acids Res. 18(3): 493-499). The peptide should be linked to the resin with a suitable linker that will be cleaved under conditions normally used for cleaving and deprotecting oligonucleotides. A 4-Hydroxymethylbenzoic acid (HMBA) linker (Sheppard et al., 1982, Int. J. Peptide Protein Res. 20: 451) is base-cleavable and suitable for both FMOC peptide synthesis and oligonucleotide synthesis. This can be readily coupled to amino-functionalised CPG beads such as aminopropyl-CPG, which is commercially available.

An example of a synthesis according to this embodiment of the invention is illustrated in FIG. 3. In FIG. 3 it can be seen that tag oligonucleotide conjugates according to this invention may be prepared by initially synthesising the peptide tag component of the conjugate on a controlled pore glass (CPG) resin that is compatible with automated oligonucleotide synthesis. In step (1) of FIG. 3, aminopropyl CPG is initially coupled to the aforementioned HMBA linker. Peptide synthesis is initiated from this linker. In step (2) of the method in FIG. 3, an O-trityl protected serine residue is coupled to the HMBA linker. Spacers, such as beta-alanine, could be introduced between the serine and the HMBA linker if desired to reduce potential steric interference from the resin. In step (3), the desired peptide tag is synthesised using commercially available FMOC-amino acids, typically using multiple cycles of FMOC peptide synthesis. In this example, an FMOC protected photocleavable linker (4-[4-(1-(FMOC-amino)ethyl)-2-methoxy-5-nitrophenoxy)butanoic acid available from IRIS Biotech GmbH, Marktredwitz, Germany) is introduced followed by FMOC-alanine, followed by 1-Fmoc-piperidin-4-ylacetic acid (Sigma Aldrich, UK), followed again by FMOC-alanine, which is finally acetylated to block the terminal amino group so that it does not interfere in subsequent oligonucleotide synthesis. Between step (3) and step (4), the CPG resin is transferred from the automated peptide synthesiser reaction vessel or column into a column or vessel suitable for use in an automated DNA synthesiser. In step (4) of FIG. 3, the trityl protection group is removed using standard deprotection conditions in the DNA synthesiser, typically trichloroacetic acid or dichloroacetic acid in dichloromethane. In step (5), an optional Dimethoxtrityl (DMTr) protected linker is introduced to reduce steric hindrance from the solid support and the peptide (Spacer Phosphoramidite 9 from Glen Research, Stirling, Va., USA). A wide variety of similar spacers is known in the art and could be substituted for the linker shown, if desired. In step (6), multiple cycles of standard automated (phosphoramidite) DNA synthesis are carried out to generate the desired DNA sequence linked to the peptide. In the final step (7), the side chain deprotection groups are cleaved along with the HMBA linker, typically using NH₃/H₂O steps, releasing the deprotected conjugate into solution. The released conjugate would usually then be purified by high performance liquid chromatography, gel filtration, gel electrophoresis or other standard techniques known in the art.

The method shown in FIG. 3 and described above conjugates the peptide tag at the 3′ end of the oligonucleotide. However, “reverse” synthesis (5′ to 3′) is also possible (see Claeboe et al., 2003, Nucleic Acids Res. 31(19): 5685-5691). Reverse synthesis phosphoramidites used with the method in FIG. 3 (and also the methods in FIGS. 4 and 5) would yield oligonucleotides modified at the 5′ terminus. Phosphoramidite monomers for 5′ to 3′ synthesis are commercially available, for example from Glen Research Corporation (Sterling, Va., USA).

Many amino acids are known in the art and not all are available as FMOC derivatives. It is, however, comparatively simple for one of ordinary skill in the art to prepare FMOC protected derivatives of unprotected amino acids using standard methods for use with this invention (Fields et al., 1990, Int J Pept Protein Res. 35(3): 161-214).

PNA peptide conjugates can be readily synthesised as both PNA and peptides can be synthesised by FMOC chemistry. Examples of such probes are disclosed in Thompson et al. (2007, Nucleic Acids Res. 35(4): e28).

To allow more than one tag to be incorporated per oligonucleotide, mass tags can be incorporated into the oligonucleotide through conjugation to thymidine analogues, for example, as disclosed in Brown et al. (2001, Tetrahedron Lett. 42: 2587-2592). In that publication, a thymidine analogue is described with a linker coupled to the purine ring of the thymidine. This thymidine analogue has a hydroxyl group protected with an FMOC group on the end of the linker that can be made available after the nucleotide has been coupled into an oligonucleotide during automated oligonucleotide synthesis to allow a phosphoramidite modified tag to be incorporated into an oligonucleotide. Since this analogue can be incorporated within the chain, multiple linkers and hence tags can be couple to the oligonucleotide.

Alternatively, branched peptides can be synthesised incorporating multiple tag peptide sequences. The branched peptides can be synthesised on CPG, with subsequent synthesis of the oligonucleotide probe (see Haralambidis et al., 1990, Nucleic Acids Res. 18(3): 501-505).

Examples of schemes according to this embodiment of the invention are shown in FIGS. 4 and 5. In FIG. 4(a), it can be seen that tag oligonucleotide conjugates according to this invention are prepared by initially synthesising the peptide tag component of the conjugate on a controlled pore glass (CPG) resin that is compatible with automated oligonucleotide synthesis. In step (1) of FIG. 4(a), aminopropyl CPG is initially coupled to the aforementioned 4-Hydroxymethylbenzoic acid (HMBA) linker. A spacer could be introduced between the aminopropyl glass and the linker of desired or a CPG resin that has a longer linker could be employed. Peptide synthesis is initiated from the HMBA linker. In step (2) of the method in FIG. 4(a), an N-alpha-Fmoc-N-epsilon-1-(4,4-dimethyl-2,6-dioxocyclohex-1-ylidene)ethyl-L-lysine (α-FMOC-ε-Dde-Lys) residue is introduced. Spacers, such as beta-alanine could be introduced between the lysine and the HMBA linker if desired to reduce potential steric interference from the resin. The Dde protection group is removable under 2% hydrazine in Dimethylformamide (DMF) (Bycroft et al., 1993, J. Chem. Soc., Chem. Commun. 778-779) and is used for the purpose of synthesising branched peptides. In step (3) of FIG. 4(a), a further α-FMOC-ε-Dde-Lys residue is introduced, followed by an α-FMOC-O-trityl protected serine residue. The final FMOC group is removed and the serine amino group is acetylated to prevent further reaction. In step (4), the Dde protection groups are removed. In step (5), the desired peptide tag is synthesised, typically using multiple cycles of peptide synthesis, from both of the free epsilon amino groups exposed by the removal of the Dde groups using commercially available FMOC-amino acids. In this example, an FMOC protected photocleavable linker (4-[4-(1-(FMOCamino)ethyl)-2-methoxy-5-nitrophenoxy)butanoic acid available from IRIS Biotech GmbH, Marktredwitz, Germany) is introduced followed by FMOC-alanine, followed by 1-Fmoc-piperidin-4-ylacetic acid (Sigma Aldrich, UK), followed again by FMOC-alanine, which is finally acetylated to block the terminal amino group so that it does not interfere in subsequent oligonucleotide synthesis. Between step (5) and step (6), the CPG resin is transferred from the automated peptide synthesiser reaction vessel or column into a column or vessel suitable for use in an automated DNA synthesiser. In step (6) of FIG. 4(a), the trityl protection group is removed using standard deprotection conditions in the DNA synthesiser, typically trichloroacetic acid or dichloroacetic acid in dichloromethane.

In step (7) shown in FIG. 4(b), an optional Dimethoxtrityl (DMTr) protected linker/spacer is introduced to reduce steric hindrance from the solid support and the peptide (here, Spacer Phosphoramidite 9 from Glen Research, Stirling, Va., USA). A wide variety of similar spacers is known in the art and could be substituted for the linker shown, if desired. In step (8), multiple cycles of standard automated (phosphoramidite) DNA synthesis are carried out to generate the desired DNA sequence linked to the peptide. In the final step (9), the side chain deprotection groups are cleaved along with the HMBA linker, typically using NH₃/H₂O steps, releasing the deprotected conjugate into solution. The released conjugate would usually then be purified by high performance liquid chromatography, gel filtration, gel electrophoresis or other standard techniques known in the art.

The number of peptides linked to a single oligonucleotide can be varied by varying the number of α-FMOC-ε-Dde-Lysine residues introduced into the peptide in the first steps of the synthesis shown in FIG. 4. If only a single tag is desired then only one α-FMOC-ε-Dde-Lysine residue need be coupled, but if 5 tags were desired this can be achieved by coupling 5 α-FMOC-ε-Dde-Lysine residues.

In principle, the order of certain steps can be varied as well. The introduction of the optional spacer in step 7 of FIG. 4 could take place prior to removal of the Dde groups. This may be desirable if many peptides or if large peptides are to be coupled to a single oligonucleotide causing steric hindrance that might reduce the efficiency of the introduction the linker.

In further variations on the method in FIG. 4, α-Dde-ε-FMOC-Lysine can be employed in place of α-FMOC-ε-Dde-Lysine. In one variation, the O-trityl serine needed to initiate oligonucleotide synthesis can be coupled at the epsilon amino while the peptide tag is synthesised at the alpha position after removal of the Dde group. Alternatively, the alpha-Dde group can be removed prior to the epsilon-FMOC group, and the O-trityl serine can be coupled at this position. Optional spacers, such as beta-alanine residues can be introduced before coupling of the O-trityl protected serine, if desired.

In the example shown in FIG. 5, it can be seen that tag oligonucleotide conjugates according to a further embodiment of the present invention are prepared by initially synthesising the peptide tag component of the conjugate on a controlled pore glass (CPG) resin that is compatible with automated oligonucleotide synthesis. In step (1) of FIG. 5, aminopropyl CPG is initially coupled to an HMBA linker. Peptide synthesis is initiated from this linker. In step (2) of the method in FIG. 5, an α-FMOC-O-trityl protected serine residue is coupled to the HMBA linker. Spacers, such as beta-alanine, could be introduced between the serine residue and the HMBA linker if desired to reduce potential steric interference from the resin. In step (3), an α-FMOC-ε-FMOC-protected Lysine residue is introduced and coupled to the serine residue. In step (4), the desired peptide tag is synthesised from both of the free amino groups exposed by the removal of the FMOC groups on the lysine amino groups. The peptide tags are synthesised using commercially available FMOC-amino acids. In this example, an FMOC protected photocleavable linker (4-[4-(1-(FMOC-amino)ethyl)-2-methoxy-5-nitrophenoxy)butanoic acid available from IRIS Biotech GmbH, Marktredwitz, Germany) is introduced followed by FMOC-alanine, followed by 1-Fmoc-piperidin-4-ylacetic acid (Sigma Aldrich, UK), followed again by FMOC-alanine, which is finally acetylated to block the terminal amino group so that it does not interfere in subsequent oligonucleotide synthesis. Between step (4) and step (5), the CPG resin is transferred from the automated peptide synthesiser reaction vessel or column into a column or vessel suitable for use in an automated DNA synthesiser. In step (5) of FIG. 5, the trityl protection group is removed using standard deprotection conditions in the DNA synthesiser, typically trichloroacetic acid or dichloroacetic acid in dichloromethane. In step (6), an optional Dimethoxtrityl protected linker is introduced to reduce steric hindrance from the solid support and the peptide (Spacer Phosphoramidite 9 from Glen Research, Stirling, Va., USA). A wide variety of similar spacers is known in the art and could be substituted for the linker shown, if desired. In step (7), multiple cycles of standard automated (phosphoramidite) DNA synthesis are carried out to generate the desired DNA sequence linked to the peptide. In the final step (8), the side chain deprotection groups are cleaved along with the HMBA linker, releasing the deprotected conjugate into solution. The released conjugate would usually then be purified by high performance liquid chromatography, gel filtration, gel electrophoresis or other standard techniques known in the art.

The number of peptides linked to a single oligonucleotide can be varied by varying the number of α-FMOC-ε-FMOC-Lysine residues introduced into the peptide in the first steps of the synthesis. After the introduction of the first α-FMOC-ε-FMOC-Lysine, removal of the FMOC groups will expose two amino groups. This means in the next cycle of synthesis 2 further α-FMOC-ε-FMOC-Lysine groups could be introduced and four in the next cycle if desired. Three cycles of coupling of α-FMOC-ε-FMOC-Lysine would make 8 amino groups available for the synthesis of peptide tags.

Hybrid methods employing the strategies of FIGS. 3 to 5 are envisaged. For example, steps (1) to (4) of FIG. 3 could be completed, i.e. up to the removal of the Dde groups, then α-FMOC-ε-FMOC lysine residues could be coupled to the amino groups exposed by removal of the Dde groups allowing further branching to be introduced if desired.

Several photocleavable linkers are commercially available. For example, the FMOC protected linker 4-[4-(1-(FMOCamino)ethyl)-2-methoxy-5-nitrophenoxy)butanoic acid is commercially available from Iris Biotech GmbH (Marktredwitz, Germany) and from Advanced ChemTech, Inc (Kentucky, USA). This can be readily incorporated into peptides during conventional FMOC synthesis.

Branched peptides (or other polyamides), with or without attached oligonucleotides, and comprising a plurality of cleavable branches form another aspect of the invention.

Non-natural amino acids may be used in this invention, including FMOC-piperazin-1-ylacetate, 1-Fmoc-piperidin-4-ylacetic acid, N,N-Dimethyl glycine, β-Dimethylamino-DL-alanine (all commercially available from Sigma Aldrich, UK).

In addition to the tags shown in FIGS. 4 and 5, exemplar peptide sequences of the invention include:

### Series 1:

Alanine-piperazin-1-ylacetic acid-Alanine,
Alanine-piperazin-1-ylacetic acid-Valine,
Valine-piperazin-1-ylacetic acid-Alanine,
Valine-piperazin-1-ylacetic acid-Valine;

### Series 2:

(SEQ ID NO: 1)
N,N-Dimethyl glycine-Alanine-Proline-Alanine,
(SEQ ID NO: 2)
N,N-Dimethyl glycine-Alanine-Proline-Valine,
(SEQ ID NO: 3)
N,N-Dimethyl glycine-Valine-Proline-Alanine,
(SEQ ID NO: 4)
N,N-Dimethyl glycine-Valine-Proline-Valine;
and

### Series 3:

(SEQ ID NO: 5)
Cysteic Acid-Alanine-Aspartic acid-Alanine,
(SEQ ID NO: 6)
Cysteic Acid-Alanine-Aspartic acid-Valine,
(SEQ ID NO: 7)
Cysteic Acid-Valine-Aspartic acid-Alanine,
(SEQ ID NO: 8)
Cysteic Acid-Valine-Aspartic acid-Valine.

In the series 1 peptides, it can be seen that the collision cleavable linker (piperazin-1-ylacetic acid) is also a charge-carrying group due to the presence of the tertiary amino group in the piperazine ring. In series 2 and 3 peptides, the charge-carrying groups are separate entities, i.e. N,N-Dimethyl glycine and cysteic acid, respectively. In alternative embodiments, the charge-carrying groups could act as the mass modifier as well, allowing the adjacent mass modifier amino acid group to be removed resulting in a smaller tag if that were desirable.

In the series 2 peptides, the proline residue can be substituted with piperidin-4-ylacetic acid. In series 2, the N,N-Dimethyl glycine can be replaced with any easily protonated positive charge-carrying group including the natural amino acids lysine and histidine, tertiary amino group containing molecules such as β-Dimethylamino-alanine. Secondary amino containing groups may also be introduced such as nipecotic acid. Pyridine containing compounds such as nicotinic acid are also appropriate.

FIG. 6a illustrates a mass tagged oligonucleotide probe based on the peptide, (N)-Acetate-Valine-piperazin-1-ylacetic acid-Alanine-Beta-alanine-(C) [SEQ ID NO: 9]. This structure is another variant of the series-1 peptides shown above. The mass tag fragment released from SEQ ID NO: 9 by photocleavage (“P”) is also shown.

FIG. 6b illustrates a set of 9 isobaric tags (with [M+H]⁺=437.3) constructed by combinatorial rearrangement of the mass modifier amino acids, beta-alanine, alanine and valine, and isotopes of acetic acid in the tags. All of the isotope-substituted reagents are commercially available (for example from Cambridge Isotope Laboratories, Inc; Andover, Mass., USA).

Large numbers of different sets of tags can be constructed by changing the amino acids and other entities used as mass modifiers. The acetate group blocking the N-terminal amino group of the peptide tags could be replaced with any carboxylic acid, e.g. propionic or butyric acids, which can be obtained with isotope substitutions (for example from Cambridge Isotope Laboratories, Inc; Andover, Mass., USA). Similarly, further isobaric compounds can be obtained by swapping the positions of the alanine and valine residues, for example. Synthesis of every combination of isotope substituted amino acids shown in this sequence would give rise to hundreds of different tags, although not all would be isobaric with each other. If every possible, isotopic variant of the sequence shown in FIG. 6b was synthesised, the tags would fall into a series of isobaric sets. Although these variants are too numerous to enumerate here, they are within the scope of the present invention.

In all these series the mass modifiers comprise individual amino acids of valine or alanine or their isotopes. However, other amino acids can be used for this purpose. In addition, more than one amino acid can be inserted into the sequence to act as mass modifiers. To ensure cleavage is most favoured at a particular site in the sequence, beta-amino acids can also be used to act as mass modifiers as these do not readily support formation of oxazolone structures and thus do not cleave as easily as alpha amino acids at any given collision energy. In FIG. 6b the combination of alanine and beta-alanine is used to act as a mass modifier. Similarly, the combination of valine and acetate are also used to act as a single mass modifier in FIG. 6b.

In an alternative embodiment, an acid cleavable linker can be used. Since most MALDI matrix materials are acidic, addition of the matrix will effect cleavage of the mass tags. A simple method for introducing an acid labile group is to include a P3′-N5′ phosphoramidate at the 5′ terminus of the oligonucleotide adjacent to the mass tag (Shchepinov et al., 2001, Nucleic Acids Res. 29(18): 3864-72).

In a further embodiment, the entire probe label complex can be desorbed, and cleavage of the tags can take place by collision using Post Source Decay in a Time-Of-Flight mass spectrometer or in the mass analyser of an ion trap instrument or in a collision cell in alternative geometries that are used with MALDI, such as the Q-TOF geometry. PNA probes with a collision cleavable linker between the tag peptide and a PNA probe have been described previously (Thompson et al., 2007, Nucleic Acids Res. 35(4): e28).

In an alternative embodiment, branched peptides may be coupled to one or more oligonucleotides using “click chemistry”. A popular and versatile “click reaction” is 1,3-cyclo-addition of azides with terminal acetylenes using a Copper catalyst at room temperature. 5′-Hexynyl Phosphoramidite is commercially available from Glen Research Corporation (Sterling, Va., USA), which permits the introduction of an alkyne into the 5′-terminus of an oligonucleotide. Similarly, Azidobutyrate N-HydroxySuccinimide Ester is also available from Glen Research. This can be used to introduce an azide group into a peptide with a free amino group. Alternatively, Fmoc-propargyl-Glycine is available from Sigma Aldrich allowing an alkyne function to be introduced into a peptide by FMOC synthesis. In this situation, Azidobutyrate N-HydroxySuccinimide Ester can be used to introduce and azide group into an amine-modified oligonucleotide. Multiple alkynyl groups can also be incorporated into oligonucleotides at internal positions allowing multiple conjugation of azide functionalised labels (Gierlich et al., 2006, Org. Lett. 8(17): 3639-42).

For the present invention, a branched peptide synthesised according to the methods shown in FIG. 4 or 5 could be produced but the trityl-serine can be omitted and the terminal amino group of the peptide can be coupled with Azidobutyrate N-HydroxySuccinimide Ester. Azide derivatised branched peptides could then be coupled to oligonucleotides with multiple alkyne groups to give a highly labeled conjugate.

A branched peptide comprising or consisting of multiple tags according to the present invention can also be conjugated to an oligonucleotide in a non-covalent manner, for example by employing biotin-avidin binding. Biotin can be introduced into a branched peptide by standard methods known in the art. In particular, a biotinylated lysine residue may be incorporated at any position in a peptide during standard FMOC synthesis using N-α-Fmoc-N-ε-biotinyl-L-lysine, which is commercially available (for example from Merck Chemicals Ltd, Nottingham, UK). With reference to the methods shown in FIGS. 3 to 5, the serine residue used for the coupling of an oligonucleotide could be readily replaced with biotinylated lysine. A variety of avidin counter-ligands for biotin are available, including monomeric and tetrameric avidin and streptavidin.

Methods for conjugating oligonucleotides to antibodies using avidin are known in the art and these can be adapted to the conjugation of branched peptides to oligonucleotides. DNA-peptide conjugates can be prepared by conjugating oligonucleotides to streptavidin. These streptavidin-oligonucleotide conjugates can then be linked to biotinylated branched peptides in the same manner as previously described for antibodies (Niemeyer et al., 2003, Nucleic Acids Res. 31(16): e90). Alternatively, antibodies can be conjugated to avidin and biotinylated oligonucleotides can be linked to the avidin complex (Sano et al., 1992, Science. 258(5079): 120-2).

Similarly, a branched peptide according to the invention can be conjugated to a peptide or protein (for example, an affinity ligand such as an antibody, or a functional fragment thereof) by chemically reacting the branched peptide with avidin (Iwai et al., 1988, Anal. Biochem. 171: 277-282). Branched peptides comprising mass tags of the present invention with a free cysteine thiol group can be conjugated to amino groups on avidin or an antibody using the reagent SMPB (Succinimidyl 4-[p-maleimidophenyl] butyrate) as described by Iwai et al. (1998, above). A cysteine with a free thiol can be introduced into the peptide shown in FIGS. 3 to 5 to replace the trityl-serine residue using standard FMOC synthesis methods.

### Multimeric Oligonucleotides

In some embodiments, signals from a bound probe molecule may be enhanced by incorporating multiple tags into the probe molecule. This can be done by coupling more than one tag to each probe molecule, for example multiple peptide tags as described above.

Additionally or alternatively, the probe molecule may comprise a multimeric oligonucleotide to which one or more tags (for example, peptide tags) are attached. Suitable examples of multimeric oligonucleotides include a branched oligonucleotide, a comb oligonucleotide and a dendrimeric oligonucleotide.

Oligonucleotide units of the multimeric oligonucleotide may be covalently linked directly to each other through phosphodiester bonds or through interposed linking agents such as nucleic acid, amino acid, carbohydrate or polyol bridges, or through other cross-linking agents that are capable of cross-linking nucleic acid or modified nucleic acid strands. The site(s) of linkage may be at the ends of the unit (in either normal 3′-5′ orientation or randomly oriented) and/or at one or more internal nucleotides in the strand.

In linear multimeric oligonucleotides, individual units are linked end-to-end to form a linear polymer. A branched oligonucleotide (for example, bDNA) may comprise two, three or more oligonucleotide units emanating from a point of origin to form a branched structure. The point of origin may be another oligonucleotide unit or a multifunctional molecule to which at least two oligonucleotide units can be covalently bound (see for example U.S. Pat. No. 5,124,246 and Horn et al., 1997b, Nucleic Acids Res. 25: 4842-4849). A comb (or “fork”) oligonucleotide may comprise an oligonucleotide unit backbone with one or more pendant oligonucleotide units. The pendant units usually depend from a modified nucleotide or other organic moiety having appropriate functional groups to which oligonucleotides may be conjugated or otherwise attached (see for example Horn et al., 1997a, Nucleic Acids Res. 25: 4835-4841). A dendrimeric oligonucleotide comprises non-oligonucleotide components to which oligonucleotides of the same or different sequences are attached (see for example Shchepinov et al., 1997, Nucleic Acids Res. 25: 4447-4454 and Shchepinov et al., 1999, Nucleic Acids Res. 27: 3035-3041).

The multimeric oligonucleotide for use in the invention may be linear, branched, or comprise a combination of linear and branched portions. There may be at least two branch points in the multimer, for example at least 3, preferably 5 to 10. The multimer may include one or more segments of double-stranded sequences.

A probe molecule comprising a multimeric oligonucleotide may include a sequence complementary to a target linked to two or more “address” sequences to which a secondary “address complement” probe may be designed to bind. This address complement probe would be coupled to a mass tag according to this invention. The probe molecule and address sequences may be in the form of a Y-shaped oligonucleotide of a structure described by Suzuki et al. (2000, Nucleic Acids Symp Ser. 44: 125-126). Alternatively, a single long probe sequence comprising a target recognition sequence and multiple address sequences may be used. Methods for the use of branched and comb oligonucleotide probes for in situ hybridisation (see Player et al., 2001, J Histochem Cytochem. 49(5): 603-12; Kenny et al., 2002, J Histochem Cytochem. 50(9): 1219-27) may be adapted for use in the present invention.

### Oligonucleotide-Tagged Probes

In certain embodiments of the invention, a polypeptide may be detected by binding of an oligonucleotide-labelled ligand. Examples of such ligands include a nucleic acid aptamer, a peptide, an antibody and a carboydrate. The binding of the oligonucleotide-labelled ligand may be detected by hybridisation of an oligonucleotide labelled with one or more mass tags of this invention. An oligonucleotide-labelled ligand in the form of an oligonucleotide-peptide conjugate used for target binding can be produced in the same way as mass tagged oligonucleotides described above.

Aptamers are folded nucleic acid ligands with high binding affinities for non-nucleic acid targets. Aptamers have a variety of advantages for use as ligands for the detection of a variety of different molecular targets: they can be synthesised in conventional oligonucleotide synthesisers or they can be generated by PCR, they can be evolved to higher levels of specificity and they can easily by linked to additional sequences for detection by secondary, labelled oligonucleotides or dendrimers (Stoltenburg et al., 2005, Anal Bioanal Chem. 383(1): 83-91; Tombelli et al., 2004, Biosens Bioelectron. 20(12): 2424-34).

An oligonucleotide-labelled ligand in the form of an antibody-oligonucleotide conjugate may be produced by methods known in the art. Intein Thioesters are useful means for coupling cysteine-derivatised oligonucleotides to polypeptides. Protein sequences of interest, such as antibody clone libraries, can be cloned into plasmids containing intein sequences. The resulting intein containing hybrid structures reacts with free thiols on an oligonucleotide in a mild reaction that is highly site-specific (Lovrinovic et al., 2005, Mol Biosyst 1(1): 64-69). A variety of bifunctional reactive linkers are also available, for example from Pierce (Pittsburgh, Pa.), and can be used to cross-link various functional groups. Pierce provides linkers for amino group to thiol coupling, amino to amino coupling and thiol to thiol coupling.

Conventional chemical coupling of oligonucleotides to antibodies is also known in the art. An antibody may be activated with maleimide and coupled to thiol derivatised oligonucleotides (Hendrickson et al., 1995, Nucleic Acids Res. 23(3): 522-9). In another approach, an aldehyde-modified antibody may be coupled to a hydrazide modified oligonucleotide (Kozlov et al., 2004, Biopolymers. 73(5): 621-30).

An oligonucleotide-antibody conjugate may alternatively be prepared by initially conjugating an oligonucleotide to streptavidin. This streptavidin-oligonucleotide conjugate can then be linked to a biotinylated antibody (Niemeyer et al, 2003, Nucleic Acids Res. 31(16): e90). Alternatively, an antibody can be conjugated to avidin, and a biotinylated oligonucleotide can be linked to the avidin complex (Sano et al., 1992, Science. 258(5079): 120-2).

It has been shown that oligonucleotide-antibody conjugates retain both the ability of the antibody to bind to its target and the ability of the oligonucleotide to base-pair normally with a complementary oligonucleotide (Kuijpers et al., 1993, Bioconjug Chem. 4(1): 94-102) and that these conjugates will bind to their targets and tissue allowing for detection following hybridisation of a labelled complementary oligonucleotide (Bos et al., 1994, Cancer Res. 54(13): 3479-86). This work employed radiolabelled oligonucleotides for the detection step but oligonucleotide-label ligands of this invention will be equally effective for detection of bound antibody conjugates.

### MS/MS and MSⁿ Analysis of Peptide Tandem Mass Spectrometric Tags

Tandem mass spectrometers allow ions with a predetermined mass-to-charge ratio to be selected and fragmented by collision induced dissociation (CID). The fragments can then be detected providing structural information about the selected ion. When peptides are analysed by CID in a tandem mass spectrometer, characteristic cleavage patterns are observed, which allow the sequence of the peptide to be determined. Natural peptides typically fragment randomly at the amide bonds of the peptide backbone to give series of ions that are characteristic of the peptide. CID fragment series are denoted aₙ, bₙ, cₙ, etc. for cleavage at the n^{th} peptide bond where the charge of the ion is retained on the N-terminal fragment of the ion. Similarly, fragment series are denoted xₙ, yₙ, zₙ, etc. where the charge is retained on the C-terminal fragment of the ion.

Trypsin and thrombin are favoured cleavage agents for tandem mass spectrometry as they produce peptides with basic groups at both ends of the molecule, i.e. the alpha-amino group at the N-terminus and lysine or arginine side-chains at the C-terminus. This favours the formation of doubly charged ions, in which the charged centres are at opposite termini of the molecule. These doubly charged ions produce both C-terminal and N-terminal ion series after CID. This assists in determining the sequence of the peptide. Generally speaking only one or two of the possible ion series are observed in the CID spectra of a given peptide. In low-energy collisions typical of quadrupole based instruments the b-series of N-terminal fragments or the y-series of C-terminal fragments predominate. If doubly charged ions are analysed then both series are often detected. In general, the y-series ions predominate over the b-series.

In general, peptides fragment via a mechanism that involves protonation of the amide backbone follow by intramolecular nucleophilic attack leading to the formation of a 5-membered oxazolone structure and cleavage of the amide linkage that was protonated (Schlosser & Lehmann, 2000, Mass Spectrom. 35: 1382-1390). FIG. 9a shows one proposed mechanism by which this sort of fragmentation takes place. This mechanism requires a carbonyl group from an amide bond adjacent to a protonated amide on the N-terminal side of the protonated amide to carry out the nucleophilic attack. A charged oxazolonium ion gives rise to b-series ions, while proton transfer from the N-terminal fragment to the C-terminal fragment gives rise to y-series ions as shown in FIG. 9a. This requirement for an appropriately located carbonyl group does not account for cleavage at amide bonds adjacent to the N-terminal amino acid, when the N-terminus is not protected and, in general, b-series ions are not seen for the amide between the N-terminal and second amino acid in a peptide. However, peptides with acetylated N-termini do meet the structural requirements of this mechanism and fragmentation can take place at the amide bond immediately after the first amino acid by this mechanism. Peptides with thioacetylated N-termini will cleave particularly easily by the oxazolone mechanism as the sulphur atom is more nucleophilic than an oxygen atom in the same position.

Fragmentation of the amide backbone of a peptide can also be modulated by methylation of the backbone. Methylation of an amide nitrogen in a peptide can promote fragmentation of the next amide bond C-terminal to the methylated amide and also favours the formation of b-ions. The enhanced fragmentation may be partly due to the electron donating effect of the methyl group increasing the nucleophilicity of the carbonyl group of the methylated amide, while the enhanced formation of b-ions may be a result of the inability of the oxazolonium ion that forms to transfer protons to the C-terminal fragment as shown in FIG. 9b. In the context of this invention thioacetylation of the N-terminus of a tag peptide can be used to enhance cleavage of the tag peptide at the next amide bond. Similarly, methylation of the nitrogen atom of an N-terminal acetyl or thioacetyl group will also enhance cleavage of the adjacent amide bond.

The ease of fragmentation of the amide backbone of a polypeptide or peptide is also significantly modulated by the side chain functionalities of the peptide. Thus the sequence of a peptide determines where it will fragment most easily. In general it is difficult to predict which amide bonds will fragment easily in a peptide sequence. This has important consequences for the design of the peptide mass tags of this invention. However, certain observations have been made that allow peptide mass tags that fragment at the desired amide bond to be designed. Proline, for example, is known to promote fragmentation at its N-terminal amide bond (Schwartz et al., 1997, Biol. Mass Spectrom. 21: 92) as fragmentation at the C-terminal amide gives rise to an energetically unfavourable strained bicyclic oxazolone structure. Aspartic acid also promotes fragmentation at its N-terminal amide bond. Asp-Pro linkages, however, are particularly labile in low energy CID analysis (Wysocki et al., 2000, J Mass Spectrom. 35(12): 1399-1406) and in this situation aspartic acid seems to promote the cleavage of the amide bond on its C-terminal side. Thus proline, and asp-pro linkages can also be used in the tag peptides of this invention to promote fragmentation at specified locations within a peptide.

A typical tandem mass spectrometer geometry is a triple quadrupole, which comprises two quadrupole mass analysers separated by a collision chamber, also a quadrupole. This collision quadrupole acts as an ion guide between the two mass analyser quadrupoles. A gas can be introduced into the collision quadrupole to allow collision with the ion stream from the first mass analyser. The first mass analyser selects ions on the basis of their mass/charge ratio which pass through the collision cell where they fragment. The fragment ions are separated and detected in the third quadrupole. Induced cleavage can be performed in geometries other than tandem analysers. Ion trap mass spectrometers can promote fragmentation through introduction of a gas into the trap itself with which trapped ions will collide. Ion traps generally contain a bath gas, such as helium but addition of neon for example, promotes fragmentation. Similarly photon induced fragmentation could be applied to trapped ions. Another favourable geometry is a Quadrupole/Orthogonal Time of Flight tandem instrument where the high scanning rate of a quadrupole is coupled to the greater sensitivity of a reflectron TOF mass analyser to identify the products of fragmentation.

Conventional “sector” instruments are another common geometry used in tandem mass spectrometry. A sector mass analyser comprises two separate “sectors”, an electric sector which focuses an ion beam leaving a source into a stream of ions with the same kinetic energy using electric fields. The magnetic sector separates the ions on the basis of their mass to generate a spectrum at a detector. For tandem mass spectrometry, a two sector mass analyser of this kind can be used where the electric sector provide the first mass analyser stage, the magnetic sector provides the second mass analyser, with a collision cell placed between the two sectors. Two complete sector mass analysers separated by a collision cell can also be used for analysis of mass tagged peptides.

### MALDI Matrix Dyes

Various compounds have been found to be useful as matrices (also referred to herein as matrix materials) for MALDI analysis of large biomolecules. Such compounds are generally characterised by a number of properties. The compounds generally have a strong extinction coefficient at the frequency of the laser used for desorption. The compounds are also able to isolate analyte molecules in a solid solution and the compounds are sufficiently volatile to rapidly sublime when exposed to laser shots in the MALDI mass spectrometer. The subliming dye should vaporise rapidly in a jet that entrains the embedded analyte molecules and for most purposes this should take place without fragmentation of the analyte (although fragmentation may sometimes be desirable if structural information about the analyte is sought). However, a matrix should not be too volatile as experiments can sometimes take several hours and the analyte/matrix co-crystal must remain stable under vacuum in the ion source for this period of time. The property of volatility to laser irradiation can be measured approximately by determining the initial velocity of analyte ions generated by the matrix. It has been observed that higher initial velocities correspond to “softer” ionisation, i.e. reduced fragmentation, (Karas & Glückmann, 1999, J Mass Spectrom. 34: 467-477) but high initial ion velocities of some matrices also correlates to rapid sublimation under vacuum.

Different matrices have different properties in terms of their ability to assist the desorption of embedded analytes and in the subsequent sensitivity with which the analytes are detected. It has been found empirically that certain matrices are more appropriate for the analysis of particular analytes than others. For example, 3-hydroxypicolinic acid has been found to be most effective for analysing oligonucleotides (Wu et al., 1993, Rapid Commun. Mass Spectrom. 7:142-146), while 2,5-dihydroxybenzoic acid and 4-hydroxy-alpha-cyano-cinnamic acid (HCCA) are both most effective for the analysis of peptides and proteins (Strupat et al., 1991, Int. J. Mass Spectrom. Ion Proc. 111: 89-102; Beavis et al., 1992, Org. Mass Spectrom. 27: 156-158). Various cinnamic acid derivatives have been found to be effective for the analysis of proteins (Beavis & Chait, 1989, Rapid Commun Mass Spectrom. 3(12): 432-435) and the choice of matrix is dependant on the nature of the analyte, for example sinnapinic acid may be preferred over HCCA for large peptides and polypeptides, while HCCA is generally preferred for smaller peptides. 2,5-dihydroxybenzoic acid may produce less fragmentation than the cinnamic acid derivatives in some cases.

Most of the matrices described above are acidic matrices. Basic matrices have also been developed and may be more appropriate for the analysis of acid-sensitive compounds (Fitzgerald et al., 1993, Anal Chem. 65(22): 3204-3211).

Infrared MALDI (IR-MALDI) is similar in principal to ultraviolet MALDI (UV-MALDI) in that analytes must be embedded in a matrix that preferably has a strong extinction coefficient at the frequency of the laser in the desorption instrument. Appropriate matrices tend to be different compounds from those used in UV-MALDI and liquid matrices are often used. Glycerol, urea, ice and succinic acid have all been shown to be effective matrices for IR-MALDI (Talrose et al., 1993, Rapid Commun Mass Spectrom. 13(21): 2191-2198). However, some UV-MALDI matrices, such as cinnamic acid derivatives, also work as IR matrices (Niu et al., 1998, J. Am. Soc. Mass Spectrom. 9: 1-7).

Liquid matrices for UV-MALDI have also been explored (Ring & Rudich, 2000, Rapid Commun Mass Spectrom. 14(6): 515-519; Sze et al., 1998, J Am Soc Mass Spectrom. 9(2): 166-174; Karas et al., 1991, Mass Spectrom Rev 10: 335). The simplest examples of liquid matrices comprise solutions of the matrices used as solids. True liquid matrices are also known such as nitrobenzoyl alcohol. Both types of matrix have some advantages in terms of sample consistency, stability under vacuum and ease of handling, although solid matrices tend to be more sensitive. In the context of the present invention, the improvements in sensitivity may justify the use of liquid matrices. This may have particular advantages in the automation of sample preparation, as liquid handling robotics are widely available and the use of solutions of matrices, for solid matrix co-crystallisation, which readily clog dispensing devices can be avoided.

### Matrix Application

Various sample preparation methods can be used to obtain signals from biological samples: rinsing the sample in saturated dihydroxybenzoic acid dissolved in highly purified water, or coating samples by spraying a solution of hydroxy-alpha-cyano-cinnamic acid or laying a section of tissue on a matrix that has been pre-coated with a matrix. For a review see Caldwell & Caprioli (2005, Mol Cell Proteomics. 4(4): 394-401). Acoustic spraying has been reported for coating tissue sections (Aerni et al., 2006, Anal Chem. 78: 827-834). Robotic spotting of matrix has also been reported (Groseclose et al., 2007, J Mass Spectrom. 42: 254-262).

Matrix can also be pre-coated onto targets (Miliotis et al., 2002, Rapid Commun Mass Spectrom. 16(2): 117-26). Tissue sections can then be laid directly onto the pre-coated targets for analysis.

### Calibrants

MALDI spectra can vary quite considerably in terms of intensity, making comparison of quantities between spectra challenging. To provide accurate maps of the intensity of a particular tag ion and thus a map of the binding of the corresponding probe, it is important to be able to make quantitative comparisons between tag ion spectra from different locations from a tissue section. To make these comparisons, the intensities of the tag ions in spectra under comparison may be normalised. Normalisation may be carried out by incorporating a known quantity of a tag or tagged probe into the sample.

In one embodiment, a tag or tagged probe for normalisation is included at a known concentration in the matrix applied to the tissue section. Alternatively or in addition, a known quantity of a spike target can be added to the tissue sample.

In some embodiments, it may be desirable to use more than one type of calibrant with the same specimen or sample.

When spectra are obtained according to the method of this invention, the intensity of the peaks of the calibrants can be used to normalise the spectra. In the simplest case, where a single calibrant ion is dissolved in the matrix, normalisation can be achieved by dividing all of the intensities of the other tag ions by the intensity of the calibrant tag ion.

With more than one calibrant ion, calibration curves can be plotted to adjust ion intensities over a wide range of intensities.

### Signal Processing

Although the methods of this invention can be practiced on any MS/MS capable instrument, the fastest MS/MS instruments are likely to be based on Time-of-Flight (TOF) technology, such as TOF/TOF, Trap/TOF and Quadrupole/TOF geometries. Speed is important to allow decent resolution images to be obtained in a reasonable amount of time.

We now describe some of the issues in analysis of TOF data. Processing of Time-Of-Flight data is usually performed by software provided by the manufacturer of the instrument, e.g. the MassLynx software provided by Micromass (Manchester, UK) to operate their ESI-TOF and Q-TOF instrumentation. It is, however, sometimes preferable to be able to process the data directly and the general steps necessary to process TOF data to render it compatible with the methods of this invention are described below. For a review of some of the standard digital signal processing techniques described below, see for example Smith, S. W. (1997, “The Scientist and Engineer's Guide to Digital Signal Processing”, California Technical Publishing).

Raw data from the TOF is obtained in the form of digital signals from an analogue to digital converter that receives ion arrival times from the detector. The first step is to convert the ion arrival time data into ion mass-to-charge ratio data using a calibration file. Typically the digital spectra from the TOF mass analyser are contaminated by low levels of random noise. Preferably, this noise is removed prior to further analysis. Various methods of removing noise are applicable. In general the noise levels are very low compared to the ion signals. The simplest noise elimination method, therefore, is to set a threshold intensity below which the signal will ignored (or removed). However, the noise level for a Time-Of-Flight mass analyser is found to vary as the mass-to-charge ratio increases so it is better to apply a varying threshold for different mass-to-charge ratios. A standard threshold function could be determined for a given instrument relating noise to the mass-to-charge ratio and this could be used to eliminate signals below the threshold level of intensity. A preferred method, however, would be to make a data-dependant noise-estimation for different mass-to-charge ratios for each spectrum, as this allows random variations between analyses on a particular instrument to be accounted for and it makes the method independent of the instrument used. This can be done by splitting the raw spectrum into bins and estimating the noise in each bin. An interpolation or spline function describing an appropriate curve can then be fitted to the noise estimates for each bin to provide an adaptive threshold that varies over the full mass-to-charge ratio range of the spectrum. Signals below the calculated threshold are then removed from the spectrum.

After the random background noise has been removed the digital signal are smoothed prior to attempting to find ion peaks in the data. Smoothing can be achieved by various methods. Typically the digital mass spectrum data would be convoluted with a low bandpass filter. A low bandpass filter generally smoothes a digital signal by effectively determining a moving average of the signal. This removes very high frequency signals from the data that correspond to small random variations in the digitised signal intensities for each ion. The digital signal can be convoluted with a number of different filter kernels that have a smoothing effect, such as a simple square function, which produces a modified spectrum in which a moving average has been applied where there is equal weighting to every point in the moving average. A preferred filter kernel applies a higher weighting to the central point in the moving average. Appropriate filter kernels include filters derived from a windowed sinc function, Blackman windows and Hamming windows. In a more preferred embodiment, the TOF spectrum is smoothed by convolution with a filter kernel derived from a Gaussian function.

Identification of peaks in a digital signal is essentially the same as for a continuous signal. With a continuous signal the first and second differentials of the signal are calculated; maxima and minima of the signal, i.e. peaks and troughs, are identified where the first differential is zero, while maxima are identified where the second differential is negative. For a discrete signal a Laplacian filter determines appropriate corresponding difference equations that facilitate detection of peaks in the digital signal.

In the context of this invention, the methods will be applied to the MS/MS spectra obtained from tag peptides. Once a list of peaks has been identified from the TOF data with their corresponding mass-to-charge ratios, the rapid analysis techniques described below can be applied to this list of peaks.

### Rapid Analysis

MALDI TOF spectra are known for poor reproducibility in terms of peak intensities (Albrethsen, 2007, Clin Chem. 53(5): 852-8). Individual spectra generated from single laser shots are usually insufficient for meaningful interpretation. Typically, a spectrum is produced by summing up between 10 and 100 individual laser shots. This means that to generate a one megapixel image from conventional spectra may require as many as 100 million laser shots, which would be extremely time-consuming.

In a further embodiment of this invention, a method of rapidly scanning a sample is provided. In the field of image encoding and compression for electronic transmission, one method for compressing an image is to only record the points in the image where the image information changes. In simple terms, an image is defined as a series of areas of the same colour and a set of instructions, which define the relative positions and sizes of these different areas. A similar approach can be taken with imaging mass spectrometry for actually generating an image. The control system for the mass spectrometer can generate a full mass spectrum for the first spot on the tissue by acquiring and summing up 10 or 20 or other suitable number of laser shots. The laser can then be focused on the next spot and a laser shot can be acquired. In principle, the shot can be compared with the first spectrum to see if there is any change from the first spectrum. If no change in peak intensities is observed the laser can be focused onto the next location on the tissue section. This can be repeated until a change in intensities is observed, i.e. the point where the image changes, so that another full spectrum can be obtained. In practice, however, making this comparison is not trivial since MALDI TOF spectra are not particularly reproducible.

As described earlier, the methods and reagents of this invention provide a means to determine spectrum quality and to compare spectra and individual shots quantitatively. The quality of each shot (or spectrum produced by summing a number of shots) can be determined by using the intensities of calibrant ions. One method to compare the quality of shots or spectra is to determine whether the intensity of the calibrant ion is above a predetermined threshold. If more than two or more calibrant ions are added to the matrix or sample in a predetermined ratio, then a second method can be used to assess spectrum or shot quality: the ratio of the intensity of the first calibrant to the second can be used to determine whether the spectrum or shot is of a good quality. If there is a small or no deviation from the expected ratio then it is reasonable to assume that the spectrum is of a better quality than a spectrum where the determined ratio deviates from the expected ratio by a substantial amount. Thus the number of shots required from each location can be varied in accordance with a measurement of the quality of the summed spectrum.

An alternative approach is to perform a survey scan, i.e. survey spectra generated from 1 shot or summation of a small number of shots (such as 2 or 3) from each location, to rapidly generate a crude image. The data system of the mass spectrometer can then use the data from adjacent spectra to determine which locations on the sample need further shots to improve the image: adjacent spectra or shots can be compared to determine whether any change has occurred in the quantities of targets that have been probed using the tagged probes of this invention. If no change (or only a small percentage change—pre-defined by the user) is detected then it can be assumed that the region of the tissue from which the image was obtained is homogeneous.

Alternatively, a survey scan can be performed and each shot can be assessed for quality using spiked calibrants as described above. Areas of the sample where the spectra give calibrant intensities below the predetermined threshold or, if more than one calibrant ion is used, areas of the sample where the measured ratios of calibrant ions deviate significantly from the expected ratios, can be sampled again until the spectra obtained from those areas are of a good quality. This method can be improved by acquiring high quality spectra, i.e. spectra with multiple shots (e.g. by summation of 10 to 100 shots), relatively frequently, for example, after every 10 or 20 locations where survey spectra are obtained.

The basis steps of a rapid method of imaging a planar sample in accordance with the invention comprises the following steps:
The planar sample is contacted with labelled probe molecules.
Unbound probes are washed from the planar sample.
The planar sample is coated with a matrix or laid on a surface, pre-coated with matrix where the matrix contains a known concentration of at least one tag spike or at least one tagged probe molecule spike.
A first location on the planar sample is sampled a number of times by a laser in a MALDI MS/MS mass spectrometer to generate a spectrum by repeating the following steps:
   1. Desorb ions from the surface of the sample
   2. Gate or trap desorbed ions to isolate ions with mass-to-charge ratios corresponding to tags of this invention.
   3. Fragment the isolated tag ions by collision induced dissociation to release characteristic daughter ions that confirm the identity of the tags and allow relative quantification of the tags.
   4. Record the intensity of the daughter ions or add the intensities of the daughter ions to the previously recorded intensities to build a composite spectrum
   5. Compare the intensity of the spike ion with the known lowest concentration with background noise and determine whether the ratio of the spike ion signal to the background noise is above a predetermined threshold.
   6. If the ratio of the spike ion signal to the background noise is above a predetermined threshold move to record the composite spectrum as final for the current location and select a new location to sample and repeat steps 1 to 7.
   7. If the ratio of the spike ion signal to the background noise is not above a predetermined threshold repeat steps 1 to 5.

In one embodiment of this aspect of the invention, the background noise is taken as the mean of the intensity of all of the ions that are detected whose mass-to-charge ratios do not correspond to fragments from the tags of this invention.

Another rapid method of imaging a planar sample in accordance with the invention comprises the following steps:
The planar sample is contacted with labelled probe molecules.
Unbound probes are washed from the planar sample
The planar samples is coated with a matrix or laid on a surface, pre-coated with matrix where the matrix contains a known concentration of at least two tag spikes or at least two tagged probe molecule spikes.
A first location on the planar sample is sampled a number of times by a laser in a MALDI MS/MS mass spectrometer to generate a spectrum by repeating the following steps:
   1. Desorb ions from the surface of the sample
   2. Gate or trap desorbed ions to isolate ions with mass-to-charge ratios corresponding to tags of this invention.
   3. Fragment the isolated tag ions by collision induced dissociation to release characteristic daughter ions that confirm the identity of the tags and allow relative quantification of the tags.
   4. Record the intensity of the daughter ions or add the intensities of the daughter ions to the previously recorded intensities to build a composite spectrum.
   5. Compare the intensity of the two spike ions with the known lowest concentrations with each other and determine the difference between the measured ratio of the spike ion signal and the expected ratio.
   6. If the absolute value of the difference between the measured ratio of the spike ion signals and the expected ratio is less than a predetermined threshold move to record the composite spectrum as final for the current location and select a new location to sample and repeat steps 1 to 7.
   7. If the absolute value of the difference between the measured ratio of the spike ion signals and the expected ratio is greater than the predetermined threshold repeat steps 1 to 5.

A further rapid method of imaging a planar sample according to the present invention comprises the following steps:
The planar sample is contacted with labelled probe molecules.
Unbound probes are washed from the planar sample
The planar samples is coated with a matrix or laid on a surface, pre-coated with matrix where the matrix contains a known concentration of at least two tag spikes according to this invention or at least two tagged probe molecule spikes.
A first location on the planar sample is sampled a number of times by a laser in a MALDI MS/MS mass spectrometer to generate a spectrum by repeating the following steps:
   1. Desorb ions from the surface of the sample
   2. Gate or trap desorbed ions to isolate ions with mass-to-charge ratios corresponding to tags of this invention.
   3. Fragment the isolated tag ions by collision induced dissociation to release characteristic daughter ions that confirm the identity of the tags and allow relative quantification of the tags.
   4. Record the intensity of the daughter ions or add the intensities of the daughter ions to the previously recorded intensities to build a composite spectrum
   5. Compare the intensity of the two spike ions with the known lowest concentrations with each other and determine the difference between the measured ratio of the spike ion signal and the expected ratio.
   6. If the absolute value of the difference between the measured ratio of the spike ion signals and the expected ratio is above a first predetermined threshold move to record the composite spectrum as final for the current location and select a new location to sample and repeat steps 1 to 7.
   7. If the absolute value of the difference between the measured ratio of the spike ion signals and the expected ratio is greater than the first predetermined threshold repeat steps 1 to 5.

Further locations on the planar sample are sampled a number of times by a laser in a MALDI MS/MS mass spectrometer to generate a spectrum by repeating the following steps:
8. Desorb ions from the surface of the sample
9. Gate or trap desorbed ions to isolate ions with mass-to-charge ratios corresponding to tags of this invention.
10. Fragment the isolated tag ions by collision induced dissociation to release characteristic daughter ions that confirm the identity of the tags and allow relative quantification of the tags.
11. Record the intensity of the daughter ions or add the intensities of the daughter ions to the previously recorded intensities to build a composite spectrum.
12. Compare the intensity of the two spike ions with the known lowest concentrations with each other and determine the difference between the measured ratio of the spike ion signal and the expected ratio.
13. If the absolute value of the difference between the measured ratio of the spike ion signals and the expected ratio is less than a second predetermined threshold move to record the composite spectrum as final for the current location and select a new location to sample and repeat steps 8 to 14.
14. If the absolute value of the difference between the measured ratio of the spike ion signals and the expected ratio is greater than the predetermined second threshold repeat steps 8 to 12.

FIG. 7 is a flow diagram illustrating, in a general form, the steps described above. “Y” in FIG. 7 denotes “yes” and “N” denotes “no”. In step 700 the specimen is contacted with probe molecules in accordance with the invention. In step 705 unbound probe molecules are removed by washing or rinsing with a buffer. In step 710, the specimen is contacted with a MALDI matrix material, as previously described. At this stage calibrants may also be introduced into the specimen. The calibrants may be part of the matrix material or may be applied in a separate step.

Step 715 is the option of performing a survey scan is available. If a survey scan is to be performed the process continues in step 720. If a survey scan is not performed the process continues at step 725. As previously described, in a survey scan, a crude image is generated by analysing each spot on the specimen based on one or a few shots (i.e. irradiations and detection of ions). Based on the crude image, a determination of where additional shots are required can be made. The additional shots are started in step 725.

In step 725 a portion of the specimen is irradiated. This results in the generation of ions that are selected using an apparatus as described with reference to FIG. 2. The ions of interest e.g. those corresponding to the mass tags (or the probes plus mass tags) and the calibrants, are selected, for example using an ion trap, quadropole, in step 730. The selected ions are fragmented by collision with a bath gas in step 735, and the fragments detected and recorded in step 740. The amounts recorded are also normalised in step 740 to allow them to be compared and combined with other sets of results. Example techniques for normalisation have already been described.

In step 745 a determination of whether that portion of the specimen needs to be reanalysed is made. This determination may be made on the basis of a survey scan, if one has been performed, or on the basis of signal to noise ratio or a ratio of calibrant ions, as already described.

If the portion is to be reanalysed, the same portion is irradiated again in step 750 and steps 730 to 745 are repeated.

If the portion of the specimen does not need to be reanalysed, the detected and normalised amounts are recorded as final for that portion in step 755. A determination is then made of whether further portions of the specimen need to be analysed or reanalysed in step 760, i.e. whether the analysis is complete. Again, this determination may be made on the basis of the results of a survey scan, or may simply be on the basis of whether a scanning process has covered all the desired portions of the specimen.

If the analysis of the specimen is complete, the process ends at step 770. If further analysis is required, a different (new) portion of the specimen is irradiated in step 765 and steps 730 to 745 subsequently repeated.

### Tissue Preparation

In general, tissue for analysis should be treated to minimise degradation or diffusion from their starting positions of molecules of interest. Rnase inhibitors, DNAse inhibitors and Protease inhibitors can be added to washing and fixing buffers to minimise or eliminate endogenous nucleases, proteases and peptidases activity from degrading target molecules.

In order to minimise sample diffusion of molecules of interest away from their starting locations, several types of sample preparation procedure can be used.

In one preferred general procedure, tissue sections are dried slowly for several hours. MALDI matrix material (also referred to herein as “matrix”) may then be applied, e.g. by electrospray addition of matrix. The chosen matrix should be electrosprayed directly onto the dried tissue slice followed by another drying step of 15 minutes in a vacuum desiccator. Alternatively, the dried tissue section can be applied to a target that has been pre-coated with matrix.

Alternatively, tissues can be fixed using one of the many techniques known in the art (Tbakhi et al., 1998, Am J. Pathol. 152(1): 35-41). Exemplar methods are fixation with neutral buffered formaldehyde or Bouin's solution as these are generally considered the best fixatives for in situ hybridisation (Weiss & Chen, 1991, J Histochem Cytochem. 39(9): 1237-1242).

### Blotting

Direct analysis of tissue has several drawbacks, including interference from signals from low molecular weight ions (MW<500 Da) of low interest such as lipids or carbohydrates, which may obscure signals from low molecular weight molecules of interest peptides in the tissue and the potential of leakage of tissue fluid to other areas of the tissue sample preparation. In an effort to maintain the precise spatial position of a molecule in a sample with that of the actual target specimen imaged, samples may be blotted onto a stable substrate for subsequent analysis.

The target may be washed with water to remove salts and other water-soluble contaminants that do not adhere to the target surface. Other wash buffers include ammonium citrate, ammonium acetate.

Various types of target surfaces may be used such as stainless steel metal targets, C-18 micro bead covered target, nitrocellulose membranes, PVDF (Polyvinylidene difluoride) membranes.

In the simplest approach, blotting may be achieved by simply placing freshly cut tissue (with or without permeabilisation) onto the target surface for a short period, 10-30 seconds and then carefully lifting off the tissue.

Tissues may be rendered permeable prior to blotting by application of an organic solvent, such as ethanol or methanol, or by application of detergents. Detergents are, however, generally not preferred for use with mass spectrometry as they tend to suppress spectra.

Nucleic acids may also be blotted onto nitrocellulose or nylon membranes. These techniques are typically applied to gel separated nucleic acids to facilitate transfer of nucleic acids to a medium on which they can be readily probed (Brown, 2001, Curr Protoc Protein Sci., Appendix 4 & 4G; Krumlauf, 1996, Methods Mol. Biol. 58: 113-28; Pellé &-Murphy, 1993, Nucleic Acids Res. 21(11): 2783-2784). The same process should be equally applicable to a tissue section. Typically, a gel (or in the context of this invention, a tissue section), is laid on the membrane. Layers of porous material, such as paper toweling are placed on top of the gel or tissue section and pressure is applied evenly to the gel/tissue section (either using suction, or by placing a stack of paper towels and a weight on top of the membrane and gel), to ensure good and even contact between gel and membrane. Buffer transfer by capillary action from a region of high water potential to a region of low water potential (usually filter paper and paper tissues) is then used to move the DNA from the gel on to the membrane; ion exchange interactions bind the DNA to the membrane due to the negative charge of the DNA and positive charge of the membrane. The membrane is then baked, i.e., exposed to high temperature (60 to 100° C.) (in the case of nitrocellulose) or exposed to ultraviolet radiation (nylon) to permanently and covalently crosslink the DNA to the membrane. The membrane can then be contacted with a probe labeled with the tags of this invention.

Proteins can also be extracted by electroblotting onto a polyvinylidene difluoride membrane after which enzymatic digestion of the proteins can take place on the membrane (Vestling & Fenselau, 1994, Biochem Soc Trans. 22(2): 547-551).

### Other Applications:

The description above has focussed on the imaging analysis of tissue sections using the methods of this invention. Other applications of the imaging techniques described include imaging of microarrays and detection of samples that have been spotted out on a planar array.

A microarray is a 2-dimensional array of nucleic acid probes designed to be complementary to target sequences in a sample. Practically speaking a microarray could comprise an array of wells on microtitre plates, for example, such that each well contains a single immobilised oligonucleotide that is a member of the array.

Alternatively, the array may be synthesised combinatorially on a glass “chip” according to the methodology of Southern or that of Affymetrix, Santa Clara, Calif. (see for example: Pease et al., 1994, PNASUSA 91: 5022-5026; Maskos & Southern, 1993, Nucleic Acids Res. 21: 2269-2270; Southern et al, 1994, Nucleic Acids Res. 22: 1368-1373) or using related ink-jet technologies such that discrete locations on the glass chip are derivatised with one member of the hybridisation array (Barbulovic-Nad et al., 2006, Crit Rev Biotechnol. 26(4): 237-59).

In a typical microarray analysis, a label is introduced into a target sample and the target sample is hybridised to the microarray. The identity and amount of each target sequence in the sample is determined by the intensity of the signal from the label that is detected at the location corresponding to the probe that is complementary to the target sequence For the purposes of this invention it is anticipated that the tags of this invention can be readily substituted for the tags used in the many methods for labelling cDNA or cRNA with fluorescent tags (Schindler et al., 2005, Anal Biochem. 344(1): 92-101; Singh et al., 2005, Am J Physiol Cell Physiol. 288(5): C1179-89; Kurn et al., 2005, Clin Chem. 51(10): 1973-81).

In particular the method disclosed by Ericsson et al. (2008, Nucleic Acids Res. 36(8): e45) is well suited to the practice of this invention. This publication discloses the use of circularising oligonucleotide probes that are amplified by linear amplification in a two stage process. The second stage of the process produces a linear amplification product covalent linked to the surface of a microarray. Captured rolling circle products at each location on the array are probed with fluorescently labelled oligonucleotides prior to detection. The fluorescently labeled oligonucleotides described in this method can be directly substituted with the probes of this invention. In such a modified microarray experiment, scanning of the array would obviously be performed in a MALDI MS/MS instrument in accordance with the methods of this invention.

### EXAMPLE 1

### Preparation of a Peptide-Tagged Oligonucleotide

An oligonucleotide probe conjugate with the structure shown in FIG. 1 was synthesised. The peptide tag sequence was:

(SEQ ID NO: 10)
(N-terminus)-Acetate-Alanine-Piperazin-1-ylacetic
acid-Alanine-Photocleavable Linker-Glutamic Acid-
Amide-(C-terminus).

The synthesis was performed using standard FMOC amino acid chemistry on a PAL resin, which releases the C-terminal amino acid as an amide in the final deprotection and cleavage step. The synthesis was performed on a custom-made synthesiser constructed from a Gilson 215 liquid handling robot configured for peptide synthesis. The peptide was purified by HPLC.

The free glutamic carboxylic acid was used to couple the peptide to an oligonucleotide with the following sequence:

(SEQ ID NO: 11)
5′-GTATGCACCTGGGCTCTTTGCAGGTCTCT-3′.

The oligonucleotide was synthesised using standard phosphoramidite procedures on an Applied Biosystems Expedite synthesiser. After completion of the oligonucleotide synthesis, an aminohexyl linker (6-(4-Monomethoxytritylamino)hexyl-(2-cyanoethyl)-(N,N-diisopropyl)-phosphoramidite) was introduced at the 5′ terminus. The amino group was deprotected and then the peptide was coupled using a published method (Zaramella et al., 2004, J Am Chem Soc. 126(43): 14029-35). The peptide was coupled using the following relative concentrations of peptide, condensing reagent and base in dimethylformamide: 20:19:43 of peptide: HBTU (O-Benzotriazole-N,N,N′,N′-tetramethyl-uronium-hexafluoro-phosphate): NMM (1-methylmorpholine) respectively with the final concentration of peptide being 0.04 M.

The conjugate was purified by HPLC. The conjugate was then analysed by MALDI MS/MS on a Shimadzu Axima Performance TOF-TOF instrument (with Mode Reflectron, Power: 66, Gate: 324.05-333.08, P. Ext. @ 500 (bin 52), 190 mV[sim=21441 mV] Profiles 1-113 Smooth Av 1). The conjugate was analysed by co-crystallisation with both 2,5-dihydroxybenzoic acid (DHB) and 4-hydroxy-alpha-cyano-cinnamic acid (HCCA). FIG. 8 shows a mass spectrum of the photocleaved and desorbed tag peptide released from the conjugate undergoing consecutive cleavage to give the expected daughter ion (see also FIG. 1).

### EXAMPLE 2

### Selection of MALDI Matrix Material for Tag Cleavage and Detection

A further peptide, hereinafter referred to as Peptide Tag 1, was synthesised. Peptide Tag 1 has the structure:

[SEQ ID NO: 12]
(N-terminus)-Acetate-Leucine-Piperazin-1-ylacetic
acid-Leucine-Photocleavable Linker-Glutamic Acid-
Amide-(C-terminus),

and is shown in FIG. 10a (top) together with its photocleavage (“P”) fragment (bottom).

Equal concentrations of Peptide Tag 1 were analysed in three different matrices: 4-hydroxy-alpha-cyano-cinnamic acid (HCCA), 2,5-dihydroxybenzoic acid (DHB) and sinapinic acid (SA). Samples of 600 pmol of Peptide Tag 1 were co-crystallised in saturated (SA—18 mg/ml; HCCA—20 mg/ml; DHB—25 mg/ml) solutions of each matrix where each matrix was dissolved in 1:1 acetonitrile/water with 0.1% Trifluoroacetic acid. 1 μl of each sample was spotted down in micro-wells on a stainless steel MALDI target and allowed to dry. The target was then analysed by MALDI MS/MS on a Shimadzu Axima Performance TOF-TOF instrument operated in MS-mode. For each matrix, a series of spectra were collected as laser power was varied. 10 shots were acquired at each of 121 locations within a square raster of 400×400 μm and summed for each matrix sample at each laser power. The intensity of the native “peptide” ion, i.e. the Peptide Tag 1 ions that have not undergone photocleavage (m/z 821.4 for [M+H]⁺ ion), and the intensity of the ion corresponding to the expected photocleavage fragment (m/z 412.3) were recorded for each matrix (see FIG. 10a for structures of precursor tag peptide ion and the photocleaved tag peptide “fragment” ion). These values are plotted in FIGS. 10b (for HCCA), 10c (for DHB) and 10d (for SA). By comparison of FIGS. 10b, 10c and 10d, DHB gives the highest overall intensity signal for the photocleavage “fragment” ions at a laser power of 90. Note that the laser power is measured in arbitrary units defined by the instrument. SA gives similar but slightly lower intensities compared to DHB, while the signal intensity from HCCA is quite low.

In the same experiment, further spectra were obtained while operating the instrument in two different MS/MS modes. The Shimadzu instrument can perform Post Source Decay (PSD) analysis and full Collision Induced Dissociation (CID) of a selected precursor ion and both methods were applied to determine the conditions that give rise to the most intense signal from the desired daughter (m/z 257.2) ion generated by fragmentation of the photocleaved fragment ion (m/z 412.3). Again laser power was varied and the intensities of parent tag ion (precursor ion selected by the instrument) and the daughter ion were recorded for each laser power. The results of this experiment are shown in FIGS. 11a, 11b and 11c for PSD and FIGS. 11d, 11e and 11f for CID. Both DHB and SA give good results in either PSD or CID modes. Again DHB appears to be a bit more sensitive than SA but both give much higher signals than HCCA. Both give the highest signals for daughter ion detection at a laser power of 90 in the PSD mode. DHB gives the highest signal in CID mode at a laser power of 90 while SA gives the highest signal in CID mode at a laser power of 100. SA was selected for further use as it gives more homogenous deposition than DHB, which is important for consistent results in imaging.

In a further experiment, the optimum concentration of SA was determined. Equimolar concentrations of Peptide Tag 1 550 pmol/μl were dissolved in SA at various concentrations in 1:1 acetonitrile:water 0.1% TFA and spotted onto a target. The samples were analysed by PSD to determine the highest daughter ion signal at a laser power of 90. It can be seen from FIG. 12 that the highest signal was obtained with a concentration of 4 mg/ml of SA. For practical purposes, however, it was found that a concentration of 6 mg/ml sinapinic acid produces more homogenous and reproducible deposition of matrix when sprayed.

### EXAMPLE 3

### Synthesis of a Set of Three Isobaric Tags and Conjugation to Oligonucleotides

Three further peptides with the structures shown in FIG. 13(a) were synthesised. These peptides give rise to three isobaric fragments after photocleavage (note Peptide Tag 4 doesn't photocleave but is synthesised to have the structure of a photocleaved peptide tag). The peptide tag sequences were:

Peptide Tag 2:
[SEQ ID NO: 13]
(N-terminus)-Acetate-¹³C₆ Leucine-Piperazin-1-
ylacetic acid-Leucine-Photocleavable Linker-
Glutamic Acid-Amide-(C-terminus);
Peptide Tag 3:
[SEQ ID NO: 14]
(N-terminus)-Acetate-Leucine-Piperazin-1-ylacetic
acid-¹³C₆ Leucine-Photocleavable Linker-Glutamic
Acid-Amide-(C-terminus);
and
Peptide Tag 4:
[SEQ ID NO: 15]
(N-terminus)-Acetate-D₃ Leucine-Piperazin-1-
ylacetic acid-D₃ Leucine-Amide-(C-terminus).

Synthesis was performed using standard FMOC amino acid chemistry on a PAL resin, which releases the C-terminal amino acid as an amide in the final deprotection and cleavage step. The synthesis was performed on a custom-made synthesiser constructed from a Gilson 215 liquid handling robot configured for peptide synthesis. The peptide was purified by HPLC.

Peptide Tags 2 and 3 were conjugated to oligonucleotides. Peptide Tag 4 was synthesised with the structure of the expected photocleavage fragment and was designed to be used as a spike that will be added to the matrix to assist with normalisation of spectra.

The free glutamic carboxylic acid on Peptide Tag 2 was used to couple to the peptide to an oligonucleotide with the following sequence:

(SEQ ID NO: 16)
5′-GTATGCACCTGGGCTCTTTGCAGGTCTCT-3′
to give the conjugate:
CONJUGATE2:
(i.e. SEQ ID NO: 13 conjugated to SEQ ID NO: 16)
PEPTIDETAG2-5′-GTATGCACCTGGGCTCTTTGCAGGTCTCT-3′.

Similarly, Peptide Tag 3 was coupled to an oligonucleotide

(SEQ ID NO: 17)
5′-TCTCTGGACGTTTCTCGGGTCCACGTATG-3′,
having a reverse sequence of SEQ ID NO: 16,

to give the conjugate:

CONJUGATE3:
(i.e. SEQ ID NO: 14 conjugated to SEQ ID NO: 16)
PEPTIDETAG3-5′-TCTCTGGACGTTTCTCGGGTCCACGTATG-3′.

The oligonucleotides were synthesised using standard phosphoramidite procedures on an Applied Biosystems Expedite synthesiser. After completion of the oligonucleotide synthesis, an aminohexyl linker (6-(4-Monomethoxytritylamino)hexyl-(2-cyanoethyl)-(N,N-diisopropyl)-phosphoramidite) was introduced at the 5′ terminus. The amino group was deprotected and then the peptide was coupled using a published method (Zaramella et al., 2004, J Am Chem Soc. 126(43): 14029-35). The peptide was coupled using the following relative concentrations of peptide, condensing reagent and base in dimethylformamide: 20:19:43 of peptide: HBTU (O-Benzotriazole-N,N,N′,N′-tetramethyl-uronium-hexafluoro-phosphate): NMM (1-methylmorpholine) respectively with the final concentration of peptide being 0.04 M.

The conjugates were purified by HPLC. An experiment was carried out to assess the relative sensitivity of Peptide Tag 4 (the matrix spike) compared to the two conjugates. To this end, both of the oligonucleotide/peptide (CONJUGATE2 and CONJUGATE3) conjugates were mixed in equimolar quantities with Peptide Tag 4. The mixture was then analysed by MALDI MS/MS on a Shimadzu Axima Performance TOF-TOF instrument using post source decay on ions with m/z 418. The conjugate was analysed by co-crystallisation with sinapinic acid (4 mg/ml as determined from example 2). FIG. 13(b) shows a mass spectrum (869 mV 297 mV 244 mV) of the desorbed and photocleaved tag peptides at m/z 418 selected for PSD undergoing consecutive cleavage to give the expected daughter ions giving 3 distinct peaks at m/z 257, 260 and 263. After laser desorption, both conjugates and the free peptide tag 4 give rise to a single ion at 418 with successive fragmentation to give the three expected daughter ions demonstrating the multiplexing capability of the Tag Peptides of this invention. The intensity of the 260 ion is slightly greater than the other two. This ion corresponds to the Peptide Tag 4, which was not conjugated and the higher intensity for this ion reflects the fact that it does not need to undergo photocleavage. Significantly, the difference in intensity between the conjugated and unconjugated peptides is not very great implying that the efficiency of the photocleavage process is high.

FIG. 13(c) shows intensity-adjusted spectra (310 mV 56 mV 52 mV) for the three tags showing that there is very little “cross-talk” or overlap between the tags from higher mass isotopes of each tag, i.e. the higher mass isotopes of the lightest tag only contribute a neglible amount to the signal for the higher mass tags and so on.

### EXAMPLE 4

### Normalisation of Signal Intensities

Quantification in MALDI mass spectrometry is complicated by matrix effects as well as the competitive nature of the ionisation process (Karas et al., 2000, J Mass Spectrom. 35(1):1-12). Some MALDI effects are illustrated in FIG. 14(a). In this figure, an image of a MALDI target is shown. The image is a map of the ionisation intensities of a series of four samples of the calcium channel blocker drug verapamil. This molecule was selected because it illustrates the issue of quantification by MALDI. The four samples shown contain the same concentration of drug but each sample differs due to the presence of different excess “background ions”. In the first sample, the drug was mixed with the phospholipid dipalmitoyl phosphatidylethanolamine (DPPE), in the second sample the drug was mixed with Dipalmitoyl Phosphatidylcholine (DPPC), in the third with cholesterol and finally a control with no background ion. The two phospholipids and cholesterol were each made up at 10 mg/ml. The Verapamil was made up at 1 mg/ml. 1 μl of drug solution was spotted down with 1 μl of each of the lipids and left to dry. The targets were then spray coated with SA matrix (6 mg/ml) and imaged by MALDI MS (m/z at 455). In the presence of different backgrounds, the intensity of the drug ions varies considerably despite the drug being present at the same concentration in all samples.

FIG. 14(b-i) shows a further image of a MALDI target. This image is a map of the ion intensities of the daughter ion from four samples with the same concentration of CONJUGATE2 (daughter ion at m/z 257 produced by fragmentation of the m/z 418 parent ion) in the presence of various background ions. Again, the intensity of the tag ion varies with the background ion. FIG. 14(b-ii) shows the same MALDI target but the image is now a map of the intensities of the daughter ion from CONJUGATE3 (daughter ion at m/z 263 produced by fragmentation of the m/z 418 parent ion), which was added to the matrix as normalisation spike. Again, the intensity varies with background. FIG. 14(b-iii) shows the effect of normalising the intensity of CONJUGATE2 by dividing this with the intensity of CONJUGATE3 from the same location. The normalised intensities of CONJUGATE2 are homogenised effectively using a normalisation tag.

### EXAMPLE 5

### Quantification of Signal Intensities

FIG. 15(b) left graph shows the results of a dilution series of CONJUGATE2 as depicted in FIG. 15(a) left graphic, while FIG. 15(b) right graph shows a similar dilution series of CONJUGATE3 as depicted in FIG. 15(a) right graphic. 4 concentrations of each conjugate were made up; 200 fmol, 1 pmol, 5 pmol and 25 pmol were diluted in 10 mg/ml BSA. 1 μl of this sample was deposited on a metal target. The target was then spray coated with SA matrix was (6 mg/ml sinapinic acid; 1:1 acetonitrile:water with 0.1% TFA). The samples were then imaged using PSD on the photocleavage product at m/z 418. The intensities of the daughter ions at m/z 257 and 263 (see FIG. 15(a), middle graphic) for were recorded from each concentration for CONJUGATE2 and CONJUGATE3 respectively. From both graphs in FIG. 15(b), it can be seen that the intensities of each tag daughter ion increase in a direct linear relationship with the concentration of the conjugates.

### EXAMPLE 6

### Hybridisation of Probe to a Target Fixed on a Tissue Section

FIG. 16 left image shows a pair of kidney tissue sections from a mouse. The kidney was sectioned and then fixed by immersion of the tissue in 10% neutral buffered formalin for 2 hours. Kidney tissue was selected as it does not express a sequence similar to HER2_TARGET below.

50 pmol of synthetic Her2 target (see below) was sprayed onto half of one of the kidney sections (shown in FIG. 16 left image, lower section, left side) using a CAMAG ATS4 automated TLC sampler (CAMAG, Switzerland).

### HER2_TARGET:

(SEQ ID NO: 18)
5′_AGGGGTTTGCTCCGGAGAGACCTGCAAAGAGCCCAGGTGCATACCT
TGGCAATCTGCATA_3′.

After application of the HER2_TARGET sequence (SEQ ID NO: 18), the tissue was post-fixed with cold (4° C.) 0.4% paraformaldehyde/Phosphate Buffered Saline for 20 minutes. In situ hybridisation (ISH) was carried out using CONJUGATE2. The following protocol was used:
1. The tissue sections were rinsed in Diethyl Pyrocarbonate (DEPC) treated H₂0.
2. The tissue sections were then immersed in Prehybridisation buffer: 50 μl of Prehybridisation buffer was pipetted onto each section (2 ml Pre-hybridisation buffer: 240 ul 5M NaCl; 200 ul 10×PE; 20 ul 10 mg/ml salmon sperm DNA; 400 ul 50% polyethylene glycol 6000; 1140 ul DEPC H₂0). A coverslip was then laid on each section to prevent evaporation. The sections were incubated at 37° C. for 1 hr. Prehybridisation buffer was removed. The prehybridisation step blocks non-specific binding sites, decreasing background.
3. Each tissue section was then exposed to Hybridisation buffer. 50 μl hybridisation buffer was pipetted onto each section (1 ml Hybridisation buffer: 120 ul 5M NaCl; 100 μl 10×PE (500 mM PIPES; 50 mM EDTA; pH adjusted to 7.2 with 1 M KOH); 100 μl 10 mg/ml salmon sperm DNA; 200 ul 50% polyethylene glycol 6000; 100 μl of CONJUGATE2 (550 pmol/μl); made up to 1 ml with DEPC H₂0). The sections were covered with coverslips and incubated at 37° C. overnight.
4. Coverslips were removed from sections by rinsing in 4×SSC.
5. The tissue sections were then washed 3 times for 10 minutes in 1×SSC (Saline Sodium Citrate; 150 mM NaCl; 15 mM Sodium Citrate) at 37° C.
6. The tissue sections were then washed twice for 1 minute in DEPC H₂O at 37° C. to remove salt prior to MALDI mass spectrometric analysis.
7. The two tissues sections were laid onto a metal target.
8. The tissue sections were then desiccated slowly.
9. Both desiccated tissue sections were then sprayed with 6 mg/ml sinapinic acid matrix (again using the CAMAG ATS4 instrument). The sections were visualised together by MALDI TOF MS/MS using PSD selecting the m/z 418 peptide tag photocleavage fragment from CONJUGATE2 as the precursor.

FIG. 16 right image shows the distribution of the daughter ion at m/z 257 produced by PSD of the 418 ion over the two kidney sections. The daughter is located to the spiked area of the tissue. Some migration of the target appears to have occurred during the post-fixing process. Endogenous RNA is unlikely to migrate in this way, as it is typically complexed with proteins and other intracellular molecules that will ensure it fixes well during the initial fixation step.

### EXAMPLE 7

### Synthesis of a Set of Two Isobaric Tags and Conjugation to Oligonucleotides

Two peptides with the structures shown in FIG. 17 were synthesised. These peptides give rise to two isobaric fragments after photocleavage. The peptide tag sequences were:

Peptide Tag 5:
(SEQ ID NO: 19; top structure in FIG. 17)
(N-terminus)-Acetate-Leucine-Piperazin-1-ylacetic
acid-Valine-Photocleavable Linker-Glutamic Acid-
Amide-(C-terminus);
and
Peptide Tag 6:
(SEQ ID NO: 20; bottom structure in FIG. 17)
(N-terminus)-Acetate-Valine-Piperazin-1-ylacetic
acid-Leucine-Photocleavable Linker-Propargyl
Glycine-Amide-(C-terminus).

The synthesis was performed using standard FMOC amino acid chemistry on a PAL resin, which releases the C-terminal amino acid as an amide in the final deprotection and cleavage step. The synthesis was performed on a custom-made synthesiser constructed from a Gilson 215 liquid handling robot configured for peptide synthesis. The peptide was purified by HPLC.

Peptide Tags 5 and 6 were conjugated to oligonucleotides with the sequence below:

(SEQ ID NO: 21)
5′-GTATGCACCTGGGCTCTTTGCAGGTCTCT-3′.

The free glutamic carboxylic acid on Peptide Tag 5 was used to couple to the peptide to an oligonucleotide with the above sequence but modified with a 5′ amino group using the same protocol as Example 3.

### CONJUGATE4:

(i.e. SEQ ID NO: 19 conjugated to SEQ ID NO: 21)
PEPTIDE TAG5-5′-GTATGCACCTGGGCTCTTTGCAGGTCTCT-3′.

Similarly, Peptide Tag 6 (SEQ ID NO: 20) was coupled to three oligonucleotides with the same sequence as SEQ ID NO: 21 using the Copper Catalysed Azide Alkyne Cycloaddition (CuAAC) reaction (Gogoi K et al., 2007, Nucleic Acids Res. 35(21):e139). Each oligonucleotide had 1, 2 or 4 internal amino groups on the second thymidine residue of SEQ ID NO: 21, to produce CONJUGATE_{—}1TAG, CONJUGATE_{—}2TAG and CONJUGATE_{—}4TAG, respectively.

The oligonucleotides were synthesised using standard phosphoramidite procedures on an Applied Biosystems Expedite synthesiser. To incorporate internal amino groups at the second thymidines of SEQ ID NO: 21, Amino-Modifier C6 dT (5′-Dimethoxytrityl-5-[N-(trifluoroacetylaminohexyl)-3-acrylimido]-2′-deoxyUridine, 3′-[(2-cyanoethyl)-(N,N-diisopropyl)]-phosphoramidite; Glen Research, Sterling, Va., USA) was incorporated. The Trifluoroacetyl group protecting the amino groups was removed during the standard final deprotection step.

The oligos were then modified to incorporate an azide function at each of the free amino groups. Azidobutyrate NHS Ester (Glen Research, Sterling, Va., USA) was used to introduce the azide to the amino groups. A solution of the azide reagent (100 equivalents) in DMSO (100 μl) was added to a solution of a purified oligo (0.2 micromole, 1 equivalent) in 0.5M sodium carbonate buffer pH 8.75 (100 μl). The reaction was left at Room Temperature for 5 hours, NAP-G25 gel filtered and then purified by reverse phase HPLC.

The reaction conditions for the coupling of the azide oligonucleotides to the propargyl peptide (Peptide Tag 6 [SEQ ID NO: 20]) depended on the number of azide sites in the oligonucleotides. For coupling of the oligonucleotides with one and two azide sites the aqueous reaction comprised: 1 equivalent of oligonucleotide, 20 equivalents of CuSO4, 200 equivalents of sodium ascorbate, 140 equivalents of tri-hydroxypropyltriazole and 50 equivalents of peptide. For the oligonucleotide with 4 azide sites using 1 equivalent of oligonucleotide, 50 equivalents of CuSO4, 500 equivalents of sodium ascorbate, 350 equivalents of tri-hydroxypropyltriazole and 80 equivalents of peptide.

To carry out the coupling, a solution of sodium ascorbate was added to a degassed tri-hydroxypropyltriazole solution, followed by CuSO4 solution, peptide solution and oligo solution, respectively. With each addition step, the reaction was degassed with argon for 1 minute. Then the reaction was left in the dark at RT for 2 hours. After the reaction was completed, the conjugates were desalted by NAP-G25 (×2) gel filtration and then purified by reverse phase HPLC.

### EXAMPLE 8

### Analysis of Multiply Labeled Probes by MALDI-TOF/TOF Mass Spectrometry

The conjugates from Example 7 were made up to a concentration of 50 pmol/μl in water. The oligonucleotide/peptide conjugates were then analysed by mixing equimolar quantities of CONJUGATE4 with the other conjugates: CONJUGATE_{—}1TAG, CONJUGATE_{—}2TAG and CONJUGATE_{—}4TAG respectively. The mixture was then analysed by MALDI MS/MS on a Shimadzu Axima Performance TOF-TOF instrument using post source decay on ions with m/z 398. All four conjugates yield [M+H]+ ions at m/z 398 after Photocleavage. CONJUGATE_{—}1TAG, CONJUGATE_{—}2TAG and CONJUGATE_{—}4TAG produce a daughter ion at m/z 257 after Post Source Decay. For comparison, CONJUGATE_{—}4 was used as an internal standard, which produces a daughter ion at m/z 243. The conjugates were analysed by co-crystallisation with sinapinic acid (20 mg/ml).

Samples were prepared in duplicate with three replicates taken from the each pair. The samples were as follows:

1. 0.5 μL CONJUGATE4, 0.5 μL water and 0.5 μL sinapinic acid (20 mg/mL)
2. 0.5 μL CONJUGATE4, 0.5 μL CONJUGATE_{—}1TAG and 0.5 μL sinapinic acid (20 mg/mL)
3. 0.5 μL CONJUGATE4, 0.5 μL CONJUGATE_{—}2TAG and 0.5 μL sinapinic acid (20 mg/m L)
4. 0.5 μL CONJUGATE4, 0.5 μL CONJUGATE_{—}4TAG and 0.5 μL sinapinic acid (20 mg/mL)

For the PSD analysis the ion gate was set such that the m/z 398 ion was selected. Typical spectra are shown in FIG. 18. It can be seen in FIG. 18 that the three ions of interest are visible. The m/z 398 parent ion and the two daughter ions at m/z 243 and m/z 257 can be detected when present.

The ion intensity counts for the three ions of interest from the replicated analyses are plotted in Table 1 below. Typical MALDI spot to spot variation can be seen from analysis to analysis, but in general the ion ratios are reproducible. Replicates of sample 4 where the oligonucleotide was conjugated to more tags show a much higher ratio of the 257 ion to the control ion at 243 indicating that conjugation of more tag to the oligonucleotide increases signal intensity as expected. Similarly, sample 3 replicates (2 tag conjugate) has a signal intensity between that of the singly labelled and quadruply labelled probes.

TABLE 1

| PSD | m/z 398 | m/z 243 | m/z 257 | Ratio of 257/243 |
|---|---|---|---|---|
| 1 | 410 | 67 | | |
| 1 | 682 | 191 | | |
| 1 | 914 | 195 | | |
| 2 | 922 | 123 | 109 | 89% |
| 2 | 653 | 105 | 69 | 66% |
| 2 | 640 | 107 | 71 | 66% |
| 3 | 567 | 71 | 39 | 55% |
| 3 | 331 | 29 | 32 | 110% |
| 3 | 263 | 29 | 35 | 121% |
| 4 | 261 | 26 | 60 | 231% |
| 4 | 646 | 55 | 114 | 207% |
| 4 | 429 | 36 | 76 | 211% |

Although the present invention has been described with reference to preferred or exemplary embodiments, those skilled in the art will recognise that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

What is claimed is:

1. A method for analysing a specimen, comprising: a) contacting the specimen with probe molecules, each of the probe molecules including one or more mass tags coupled to each probe molecule via a first cleavable linker, and allowing probe molecules to become bound to the specimen; b) removing unbound probe molecules from the specimen; c) contacting the specimen with a Matrix-assisted Laser Desorption ionisation (MALDI) matrix material, or laying the specimen on a surface coated with a MALDI matrix material; d) irradiating a portion of the specimen with a laser beam to release ions from the specimen; e) selecting, from the released ions (d), ions with a mass-to-charge ratio corresponding to the mass tags or derivatives thereof; f) recording the amount and type of selected ions, together with the location of the portion of the specimen as a result.

2. The method according to claim 1, wherein (d) to (f) are repeated a plurality of times for at least one portion of the specimen.

3. The method according to claim 1, wherein the step of recording comprises combining the amount of ions selected in step (e) with a previously recorded amount of selected ions for that portion of the specimen to produce a composite result.

4. The method according to claim 1, wherein the specimen is a biological specimen.

5. The method according to claim 1, wherein the specimen is a tissue sample, an array of tissue samples or a microarray of tissue samples.

6. The method according to claim 1, wherein the specimen is a plurality of samples blotted onto a substrate from a tissue sample.

7. The method according to claim 1, wherein the specimen comprises a sample or samples labelled with oligonucleotide-labelled ligands, wherein the probe molecules bind to the oligonucleotide-labelled ligands.

8. The method according to claim 1, wherein the specimen is contacted with a plurality of different probe molecules, each different probe molecule labelled with a different mass tag.

9. The method according to claim 8, wherein at least two of the different mass tags are isotopes of one another.

10. The method according to claim 8, wherein at least two of the different mass tags are of equal mass.

11. The method according to claim 1, wherein at least one of the probe molecules comprises a plurality of mass tags.

12. The method according to claim 1, wherein the released ions comprise probe molecule-mass tag complexes and selecting includes splitting the mass tags from the probe molecules.

13. The method according to claim 12, wherein the mass tags are split from the probe molecules by photoillumination, collision or by a chemical reagent.

14. The method according to claim 11 wherein at least some of the mass tags comprise at least two mass modifiers coupled together via a second cleavable linker.

15. The method according to claim 14, further comprising: fragmenting the ions selected in (e) to produce daughter ions prior to (f); wherein (f) comprises detecting and recording the amount and type of each daughter ion together with the location of the portion of the specimen.

16. The method according to claim 15, wherein the second cleavable linker is a collision cleavable linker.

17. The method according to claim 1, wherein the first cleavable linker is a photocleavable linker.

18. The method according to claim 5, wherein the specimen is a planar tissue sample.

19. The method according to claim 1, further comprising raster scanning the specimen with the laser beam.

20. The method according to claim 1, further comprising optimising the power of the laser beam prior to (d) to provide the largest amount of a predetermined selected ion or ions selected in (e).

21. The method according to claim 1, further comprising optimising the concentration of the MALDI matrix material to provide the largest amount of selected ions for a predetermined MALDI matrix material and a predetermined laser power.

22. The method according to claim 1, further comprising the step of selecting a concentration of the MALDI matrix material that provides the most homogenous deposition of the MALDI matrix material on the specimen using a spraying operation.

23. The method according to claim 1, further comprising the step of normalising the amount of recorded selected ions.

24. The method according to claim 23, wherein normalising comprises: including in the MALDI matrix material or specimen a predetermined concentration of calibrant that produces calibrant ions when irradiated by the laser beam; detecting and recording an amount of calibrant ions produced following irradiating; and dividing the amount of ions selected in (e) by the amount of calibrant ions to provide a normalised amount of selected ions.

25. The method according to claim 23, wherein normalising comprises: including in the MALDI matrix material or specimen a predetermined concentration of a plurality of different calibrants that produce calibrant ions when irradiated by the laser beam; detecting and recording an amount of each type of calibrant ion produced following irradiating and producing a calibration function from the amounts; and calibrating the amount of each type of selected ion with the calibration function to provide a normalised amount of each type of selected ion.

26. The method according to claim 24, wherein at least one calibrant is a mass tag calibrant or a probe molecule calibrant.

27. The method according to claim 26, wherein the at least one calibrant is an isotope of one of the mass tags of the probe molecules.

28. The method according to claim 1, wherein recording further comprises producing digital signals indicative of the amount and type of selected ions, removing noise from the digital signals by removing all signals below a threshold level of recorded intensity, wherein the threshold level is dependent on the mass to charge ratio of the recorded ions to which the signals correspond.

29. The method according to claim 28, further comprising smoothing the digital signal.

30. The method according to claim 1, further comprising: analysing results of said method and determining, based on the results, whether to repeat (d) to (f) for a portion of the specimen.

31. The method according to claim 30, wherein determining comprises calculating a difference value based on the difference between the recorded result for that portion and the recorded result for at least one adjacent portion, and comparing the difference value with a predetermined threshold.

32. The method according to claim 30, wherein determining comprises determining a measure of the quality of the result based on an amount of detected calibrant ions and comparing the measure with a predetermined threshold.

33. The method according to claim 32, wherein at least one calibrant is a mass tag calibrant or a probe molecule calibrant.

34. The method according to claim 33, wherein the at least one calibrant is an isotope of one of the mass tags of the probe molecules.

35. The method according to claim 1, comprising performing (a) to (g) to produce a survey scan, and selecting portions of the specimen on which to repeat (d) to (f) based on an analysis of the survey scan.

36. The method according to claim 35, wherein irradiating is performed using a first laser spot size to produce a survey scan, and wherein irradiating is performed using a second laser spot size on selected portions of the specimen, wherein the second laser spot size is smaller than the first laser spot size.

37. The method according to claim 1, further comprising: including in the MALDI matrix material or specimen a predetermined concentration of at least one calibrant that produces calibrant ions when irradiated by the laser beam; detecting and recording an amount of the calibrant ions produced following irradiating, comparing the amount of calibrant ions with a detected background noise to provide a ratio; determining, prior to (g), whether the ratio is above a predetermined threshold; and if the ratio is above a predetermined threshold, recording the result as final for that portion of the specimen, and if the ratio is not above the predetermined threshold, returning to (d) for the same portion of the specimen.

38. The method according to claim 37, wherein a plurality of different calibrant ions are added to the MALDI matrix material or specimen, and comparing comprises comparing the amount of calibrant ions of the calibrant with the lowest detected concentration with the background noise to provide the ratio.

39. (canceled)

40. The method according to claim 37, wherein at least one calibrant is a mass tag calibrant or a probe molecule calibrant.

41. The method according to claim 40, wherein the at least one calibrant is an isotope of one of the mass tags of the probe molecules.

42. The method according to claim 41, further comprising producing an image of the distribution of each type of selected ion across the specimen.

43. A probe molecule comprising a multimeric polyamide, the multimeric polyamide having two or more polyamide branches, wherein at least two of the polyamide branches comprise or consist of a mass tag.

44. The probe molecule according to claim 43, wherein one or more or all of the polyamide branches comprising or consisting of a mass tag have a cleavable linker via which the polyamide branches are cleavable from the branched polyamide.

45. The probe molecule according to claim 44, wherein the cleavable linker is photocleavable, collision cleavable and/or acid cleavable.

46. The probe molecule according to claim 44, wherein at least one of the mass tags comprise a first mass modifier sequence coupled to a second mass modifier sequence via the cleavable linker.

47. The probe molecule according to claim 46, wherein the first mass modifier sequence, the second mass modifier sequence and/or the cleavable linker comprise a charge-carrying entity.

48. The probe molecule according to claim 47, wherein one or more or all of polyamide branches comprise a charge-carrying entity.

49. The probe molecule according to claim 48, wherein the polyamide branches are substantially identical to each other.

50. The probe molecule according to claim 49, wherein the polyamide is a peptide.

51. The probe molecule according to claim 43, wherein the polyamide is coupled to a protein and/or an oligonucleotide.

52. The probe molecule according to claim 51, wherein the oligonucleotide is coupled to the multimeric polyamide via a covalent bond.

53. The probe molecule according to claim 51, wherein the oligonucleotide is coupled to the multimeric polyamide via a non-covalent bond.

54. The probe molecule according to claim 51, wherein the oligonucleotide is up to about 100 nucleotides in length, for example about 30-60 or about 35-50 or about 40 nucleotides in length.

55. A set of probe molecules comprising at least two probe molecules as defined in claim 43, wherein the mass tags are isobaric.

56. A multimeric polyamide comprising a plurality of cleavable polyamide branches.

57. The multimeric polyamide according to claim 56, wherein at least one of the cleavable polyamide branches comprise a mass tag.

58. The multimeric polyamide according to claim 56, wherein at least one of the cleavable polyamide branches comprise a charge-carrying entity.

59. The multimeric polyamide according to claim 56, wherein the cleavable peptide branches are photocleavable, collision cleavable or acid cleavable.

60. The multimeric polyamide according to claim 56, wherein the polyamide is a peptide.

61. A probe molecule comprising a multimeric oligonucleotide, wherein a first oligonucleotide sequence of the multimeric oligonucleotide binds to a predetermined target and a second oligonucleotide sequence of the multimeric oligonucleotide is linked to a mass tag.

62. The probe molecule according to claim 61, further comprising an intermediate oligonucleotide covalently or non-covalently coupled to the mass tag, the intermediate oligonucleotide being hybridisable to the second oligonucleotide sequence thereby linking the mass tag to the second oligonucleotide sequence.

63. The probe molecule according to claim 61, wherein the second oligonucleotide sequence is covalently or non-covalently coupled to the mass tag.

64. The probe molecule according to claim 61, wherein the mass tag is cleavable from the probe molecule.

65. The probe molecule according to claim 64, wherein the mass tag is photocleavable, collision cleavable or acid cleavable.

66. The probe molecule according to claim 61, wherein the multimeric oligonucleotide is a branched oligonucleotide, a comb oligonucleotide or dendrimeric oligonucleotide.

67. The probe molecule according to claim 61, wherein the predetermined target and the mass tag are located on different branches of the multimeric oligonucleotide.

68. The probe molecule according to claim 61, wherein the mass tag is a polyamide.

69. The probe molecule or multimeric polyamide according to claims 43, wherein the polyamide is a peptide comprising at least one of the sequences: Alanine-piperazin-1-ylacetic acid-Alanine, Alanine-piperazin-1-ylacetic acid-Valine, Valine-piperazin-1-ylacetic acid-Alanine, Valine-piperazin-1-ylacetic acid-Valine, SEQ ID NOs 1-10, 12-15 and 19-20, the peptides shown in FIGS. 4 and 5, and modifications thereof.

70. The probe molecule or multimeric polyamide according to claim 43, adapted for use in mass spectrometry (MS).

71. A probe molecule or multimeric polyamide according to claim 70, in which the probe molecule or multimeric polyamide comprises an oligonucleotide sequence which binds to a target oligonucleotide sequence of an oligonucleotide-labelled ligand.

72. The probe molecule or multimeric polyamide according to claim 71, in which the oligonucleotide-labelled ligand is a nucleic acid aptamer, a peptide, an antibody or a carbohydrate capable of binding to a specimen target.

73. (canceled)

74. A kit comprising a plurality of probe molecules or multimeric polyamides according to claim 43.

75. (canceled)
